(19) United States
(12) Patent Application Publication
(10) Pub. No.: US 2013/0116148 A1
(43) Pub. Date: May 9, 2013

(54) LIVER DISEASE MARKER, METHOD AND APPARATUS FOR MEASURING THE SAME, AND METHOD FOR ASSAYING PHARMACEUTICAL PREPARATION

(71) Applicants: Tomoyoshi Soga, Tsuruoka-shi (JP); Masahiro Sugimoto, Tsuruoka-shi (JP); Makoto Suematsu, Tokyo (JP); Takafumi Saito, Yamagata-shi (JP); Sumio Kawata, Yamagata-shi (JP)

(72) Inventors: Tomoyoshi Soga, Tsuruoka-shi (JP); Masahiro Sugimoto, Tsuruoka-shi (JP); Makoto Suematsu, Tokyo (JP); Takafumi Saito, Yamagata-shi (JP); Sumio Kawata, Yamagata-shi (JP)

(73) Assignee: KEIO UNIVERSITY, Tokyo (JP)

(21) Appl. No.: 13/805,170

(22) PCT Filed: May 17, 2011
(86) PCT No.: PCT/JP2011/061340

§ 371 Date: Jan. 18, 2013

(30) Foreign Application Priority Data
Jun. 18, 2010 (JP) 2010-139736

(51) Int. Cl. G01N 33/68

(52) U.S. Cl. 506/9; 506/18; 506/39; 506/12

(57) ABSTRACT

Disclosed is a method for promptly identifying a liver disease. A normal person or a liver disease such as drug-induced liver injury, asymptomatic hepatitis B carrier, chronic hepatitis B, hepatitis C with persistently normal ALT, chronic hepatitis C, cirrhosis type C, hepatocellular carcinoma, simple steatosis, or non-alcoholic steatohepatitis is identified by measuring the concentration of a γ-Glu-X (wherein X represents an amino acid or an amine) peptide or the level of AST or ALT in blood and carrying out, for example, a multiple logistic regression based on the measured value.

### TECHNICAL FIELD

The present invention relates to a liver disease marker, a method and an apparatus for measuring the same, and a method for assaying a pharmaceutical preparation. In particular, the present invention relates to a liver disease marker that allows for screening to distinguish patients with various liver diseases from normal persons, a method and an apparatus for measuring the same, and a method for assaying a pharmaceutical preparation by using the liver disease marker.

### BACKGROUND ART

There are various types of liver diseases, such as drug-induced liver injury, hepatitis B, hepatitis C, hepatic cirrhosis, and hepatocellular carcinoma. There are also asymptomatic carriers of a B-type virus or a C-type virus. In particular, 70 percent of hepatitis C virus (HCV)-infected individuals experience gradual loss of normal stem cells, fibrosis of the liver, progression to hepatic cirrhosis, and furthermore development of hepatocellular carcinoma, due to chronic liver inflammation (chronic hepatitis). It is reported that 10 to 15% of chronic hepatitis C patients and 80% of hepatic cirrhosis patients develop hepatocellular carcinoma. Although the state of chronic hepatitis is not life-threatening, life is threatened when hepatocellular carcinoma develops or hepatic cirrhosis progresses to cause hepatic failure. Therefore, it is necessary to diagnose hepatitis C at an early stage and disinfect the virus.

Hepatitis C progresses from hepatic cirrhosis to hepatocellular carcinoma with no symptoms, and liver function deteriorates extremely resulting in various disorders such as malaise, jaundice, and disturbed consciousness. However, at this stage, there is currently no effective therapy. Therefore, it is necessary to detect progression of the symptoms as early as possible before liver function deteriorates and to apply a treatment such as interferon administration. However, there is currently no established method for identifying various liver injuries precisely and rapidly.

Generally, when a liver disease is suspected, liver function markers such as aspartate aminotransferase (AST), alanine aminotransferase (ALT), γ-glutamic pyruvic transaminase (GPT), alkaline phosphatase (AL-P), choline esterase (ChE), and bilirubin in blood are measured in conjunction with a medical examination by interview, an inspection, and a palpation. When an abnormality was found in these biochemical values, a hepatitis B virus test and a hepatitis C virus test, and imaging tests such as an ultrasound examination, an X-ray examination, and a CT examination are performed. For determination of cancer, proteinous tumor markers such as α-fetoprotein (AFP), abnormal prothrombin (PIVKA-II), and carcinoembryonic antigen (CEA) in blood are measured. Furthermore, when an accurate determination is required, laparoscopy, liver biopsy, and the like (about one week of hospital stay is required) are performed (Non Patent Literature 1).

Thus, identification of liver diseases requires many examinations and it takes many days for the disease to be determined. Laparoscopy, liver biopsy, and the like also endanger patients and cause them physical pain. Since laparoscopy, liver biopsy, and the like put a heavy burden on patients, they may not be performed frequently to check the patients' pathological conditions. Furthermore, in the case of conventional methods, many of the examinations or determinations may be performed by only experts, and therefore burden is imposed on insufficient health care practitioners. Therefore, a method for determining a liver disease rapidly, precisely, and conveniently without putting a burden on patients is highly desirable.

Many liver injuries such as hepatitis, hepatic cirrhosis, and hepatocellular carcinoma are known to be caused by generation of active oxygen (oxidative stress) and disruption of the protection system of a living organism to remove it (Non Patent Literature 2). One of the major protection systems of a living organism against oxidative stress such as active oxygen is a glutathione system. Reduced glutathione (GSH: referred to as glutathione hereinbelow) is an antioxidant that exists in the highest concentration in a tissue. Glutathione conjugates to active oxygen, electrophiles, and the like, and reduces these substances, thereby suppressing oxidative stress.

However, when the glutathione is decreased, a tissue, a cell, and the like are exposed to oxidative stress, and various pathological conditions are caused (Non Patent Literature 3). In fact, it is reported that, in liver injuries, oxidative stress is increased by infection with a hepatitis B virus or a hepatitis C virus, and glutathione is decreased, and that, in patients and mice with hepatitis C, hepatic cirrhosis, or hepatocellular carcinoma, glutathione is decreased (Non Patent Literatures 2 and 4).

Drug-induced liver injury, which is induced by taking a drug, is also caused by oxidative stress. Acetaminophen (APAP), which is an antipyretic analgesic, is metabolized in the liver to generate a highly toxic electrophile, N-acetylbenzoquinoneimine (NAQPI). This NAQPI is conjugated to by glutathione (GSH), which exists in a high concentration in the liver, and is detoxified and excreted. However, when the electrophiles exist in large quantities, the glutathione is depleted, and the electrophiles accumulate in cells (oxidative stress) and react with a biopolymer. It is known that cellular functions are consequently disturbed, thereby causing pathological conditions such as drug-induced liver injury.

Previously, the present inventors have found that glutathione was decreased in order to detoxify electrophiles, NAQPIs, generated by metabolism of APAP, and ophthalmic acid was increased rapidly in inverse proportion to the glutathione level, when large quantities of APAP were administered to a mouse (see FIG. 1(B)). The present inventors also have found that an increase of ophthalmic acid in the liver and blood indicates depletion of glutathione in the liver caused by the electrophiles (Patent Literature 1, Non Patent Literature 5).

The mechanism is as follows. As shown in FIG. 1, glutathione (γ-Glu-Cys-Gly) and ophthalmic acid (γ-Glu-2AB-Gly) are tripeptides biosynthesized by the same two enzymes, a γ-glutamylcysteine synthetase and a glutathione synthetase. They are different in their substrates (starting materials), which are cysteine (Cys) and 2-aminobutyric acid (2AB). In the normal reduction state shown in FIG. 1(A), glutathione exists in large quantities in the liver and the first enzyme, γ-glutamylcysteine synthetase is under feedback (FB) inhibition.

Therefore, little ophthalmic acid is biosynthesized. However, when electrophiles, active oxygen species, and the like exist in such a case as an oxidation state shown in FIG. 1(B), glutathione is consumed for detoxication. Feedback inhibition is canceled due to the decrease of glutathione, γ-glutamylcysteine synthetase is activated, and glutathione and ophthalmic acid are biosynthesized. Ophthalmic acid accumulates in the liver and also is excreted into the blood. As described above, since ophthalmic acid in the liver, blood, and the like increases under an oxidation state caused by an electrophile and the like, ophthalmic acid serves as a biomarker of oxidative stress.

Further, nonalcoholic fatty liver diseases (NAFLD) occur when visceral fat increases because of obesity. With respect to nonalcoholic fatty liver diseases (NAFLDs), it is also reported that serum thioredoxin (TRX), a marker of oxidative stress, is useful for distinguishing between nonalcoholic steatohepatitis (NASH) that progresses to hepatic cirrhosis and further to hepatocellular carcinoma and simple steatosis (SS) that has a favorable course, (Non Patent Literature 6).

On the other hand, there is a comprehensive method for measuring metabolites in a cell, which is based on a method of measuring metabolites in a sample by a capillary electrophoresis-mass spectroscope (CE-MS) (for example, see Non Patent Literatures 7 to 9). This comprehensive method includes determining a low molecular weight compound (metabolite) pattern and/or a peptide pattern of a liquid sample derived from a human body or an animal body qualitatively and/or quantitatively in order to monitor the condition of the human body or the animal body, wherein the metabolite and the peptide in the liquid sample are separated by capillary electrophoresis, subsequently directly ionized, and then detected on a mass spectrometer connected on-line through the interface. The reference value and the sample value that show the condition, and the deviation and correspondence derived from the values are automatically stored in a database in order to monitor the condition of the human body or the animal body over a prolonged period. When an anionic compound is separated and analyzed by combining capillary electrophoresis and mass spectrometry, a method for separating and analyzing an anionic compound including reversing electroosmotic flow by using a coated capillary whose inner surface is pre-coated cationically is known (for example, see Patent Literature 2).

### CITATION LIST

Patent Literature
Patent Literature 1: Japanese Patent Application Laid-Open No. 2007-192746
Patent Literature 2: Japanese Patent No. 3341765

Non Patent Literature
Non-Patent Literature 1: Callewaert, N. et al. Nat. Med. 10, 429-434, 2004.
Non-Patent Literature 2: Loguercio, Carmela et al. Free Radic. Biol. Med. 34, 1-10, 2003.
Non-Patent Literature 3: Yadav, Dhiraj et al. Am. J. Gastroenterol. 97, 2634-2639, 2002.
Non-Patent Literature 4: Moriya, K. et al. Cancer Res. 61, 4365-4370, 2001.
Non-Patent Literature 5: Soga, T. et al. J. Biol. Chem. 281, 16768-16776, 2006.
Non-Patent Literature 6: Kyuichi Tanikawa eds., “Sanka Storesu to Kanshikkan Vol. 5 (in Japanese) (Oxidative Stress and Liver Diseases Vol. 5)” Medical Tribune, Inc., pages 3-37, 2009. 5. 7.
Non-Patent Literature 7: Soga, T. et al. J. Proteome Res. 2. 488-494, 2003.
Non-Patent Literature 8: Soga, T. et al. J. Boil Chem. Vol. 281, No. 24 (Jun. 16, 2006), 16768-16776
Non-Patent Literature 9: Hirayama A. et al. Cancer. Res. 69: (II). (Jun. 1, 2009) 4918-4925
Non-Patent Literature 10: Pignatelli, B. et al. Am. J. Gastroenterol. 96, 1758-1766, 2001.

### SUMMARY OF INVENTION

### Technical Problem

However, it has been difficult so far to distinguish and identify drug-induced liver injury (DI), asymptomatic hepatitis B carriers (AHB), chronic hepatitis B (CHB), hepatitis C with persistently normal ALT (CNALT), hepatitis C virus carriers with a normal ALT value, and chronic hepatitis C (CHC), cirrhosis type C (CIR), hepatocellular carcinoma (HCC), non-alcoholic steatohepatitis (NASH), simple steatosis (SS), and the like in one examination.

The present invention was achieved to solve the above-mentioned conventional problems. It is an object of the present invention to enable one to identify rapidly liver diseases such as drug-induced liver injury (DI), asymptomatic hepatitis B carriers (AHB), chronic hepatitis B (CHB), hepatitis C virus carriers with persistently normal ALT (CNALT), chronic hepatitis C (CHC), cirrhosis type C (CIR), hepatocellular carcinoma (HCC), nonalcoholic steatohepatitis (NASH), and simple steatosis (SS) by measuring a low molecular weight biomarker in blood.

### Means for Solving the Problems

Since many liver injuries such as hepatitis, hepatic cirrhosis, and hepatocellular carcinoma are closely related to oxidative stress as described above, the concentration of ophthalmic acid was expected to vary in the liver injuries. Thus, blood was collected from a normal person (i.e., a control: C) and patients with drug-induced liver injury (DI), an asymptomatic hepatitis B carrier (AHB), patients with chronic hepatitis B (CHB), hepatitis C virus carriers with persistently normal ALT (CNALT), patients with chronic hepatitis C (CHC) patients with cirrhosis type C (CIR), patients with hepatocellular carcinoma (HCC), patients with non-alcoholic steatohepatitis (NASH) and patients with simple steatosis (SS), and ophthalmic acid in the sera was measured. However, unlike mice, little ophthalmic acid was detected in the normal person (C), the drug-induced liver injury (DI) patient, and the like. (While the concentration of ophthalmic acid in the murine serum was approximately 2 μM, its concentration in the human serum was approximately one twentieth the concentration in the murine serum and little ophthalmic acid was detected in the normal person (C), the drug-induced liver injury (DI) patient, and the like).

However, the present inventors discovered substances that increased significantly in the sera of the hepatitis patients and identified the substances as γ-Glu-X peptides (notes: X represents an amino acid or an amine). FIG. 2 is a pattern diagram which shows a mechanism in which γ-Glu-X peptides are biosynthesized in patients with various types of liver disorders. Furthermore, the present inventors successfully distinguished patients with various types of hepatitis from patients with other diseases by performing multivariate analysis using a multiple logistic regression model including the levels of AST and ALT, which are liver function markers in serum, and γ-Glu-X peptides.

This discovery enabled rapid identification of a normal person (C) and liver diseases such as drug-induced liver injury (DI), an asymptomatic hepatitis B carrier (AHB), chronic hepatitis B (CHB), hepatitis C virus carriers with persistently normal ALT (CNALT), chronic hepatitis C (CHC), cirrhosis type C (CIR), hepatocellular carcinoma (HCC), simple steatosis (SS), and non-alcoholic steatohepatitis (NASH) by measuring the concentrations of γ-Glu-X peptides and the levels of AST and ALT in blood.

The present invention was achieved based on the above-mentioned finding. The present invention provides a liver disease marker for detecting an oxidative stress in a mammalian tissue, wherein the marker is a γ-Glu-X (X represents an amino acid or an amine) peptide. Here, a plurality of combinations of γ-Glu-X (X represents an amino acid or an amine) peptides can be selected by multiple logistic regression (MLR) analysis.

The present invention also provides a liver disease marker for identifying a normal person (C), wherein the above-mentioned liver disease marker is a combination including at least glucosamine, γ-Glu-Ala, methionine sulfoxide, γ-Glu-Leu, γ-Glu-Val, AST, ALT, γ-Glu-Phe, γ-Glu-Met and γ-Glu-Gln, as apparent from Table 2 given later.

The present invention also provides a liver disease marker for identifying a drug-induced liver injury (DI) which is a combination including at least γ-Glu-Taurine, γ-Glu-Leu, γ-Glu-Glu, γ-Glu-Arg, γ-Glu-Ser, γ-Glu-Phe, γ-Glu-Met and γ-Glu-Citrulline excluding AST, ALT and γ-Glu-Gly which are close to 1 in odds ratio, as apparent from Table 2 given later. It is also possible to raise the accuracy by adding at least one of AST, ALT and γ-Glu-Gly.

The present invention also provides a liver disease marker for identifying an asymptomatic hepatitis B carrier (AHB) which is a combination including at least γ-Glu-Taurine, γ-Glu-Ala, γ-Glu-Leu, γ-Glu-Val, AST, γ-Glu-Lys, γ-Glu-Arg, γ-Glu-Met and γ-Glu-Gln excluding ALT which is close to 1 in odds ratio, as apparent from Table 2 given later. It is also possible to raise the accuracy by adding ALT.

The present invention also provides a liver disease marker for identifying chronic hepatitis B (CHB) which is a combination including at least γ-Glu-Ala, methionine sulfoxide, γ-Glu-Leu, γ-Glu-Glu, AST, ALT, γ-Glu-Arg, γ-Glu-Ser, γ-Glu-His, γ-Glu-Phe, γ-Glu-Met and γ-Glu-Citrulline, as apparent from Table 3 given later.

The present invention also provides a liver disease marker for identifying a hepatitis C virus carrier with persistently normal ALT (CNALT) which is a combination including at least glucosamine, γ-Glu-Leu, γ-Glu-Val, AST, γ-Glu-Gly, γ-Glu-Gln and γ-Glu-Citrulline excluding ALT which is close to 1 in odds ratio, as apparent from Table 3 given later. It is also possible to raise the accuracy by adding ALT.

The present invention also provides a liver disease marker for identifying chronic hepatitis C (CHC) which is a combination including at least glucosamine, γ-Glu-Lys and γ-Glu-His excluding methionine sulfoxide and ALT which are close to 1 in odds ratio, as apparent from Table 3 given later. It is also possible to raise the accuracy by adding methionine sulfoxide and/or ALT.

The present invention also provides a liver disease marker for identifying cirrhosis type C (CIR) which is a combination including at least glucosamine, methionine sulfoxide, γ-Glu-Leu, γ-Glu-Val, γ-Glu-Glu, γ-Glu-Gly, γ-Glu-Met, γ-Glu-Gln and γ-Glu-Citrulline excluding AST and ALT which are close to 1 in odds ratio, as apparent from table 4 given later. It is also possible to raise the accuracy by adding AST and/or ALT.

The present invention also provides a liver disease marker for identifying hepatocellular carcinoma (HCC) which is a combination including at least γ-Glu-Taurine, γ-Glu-Glu, γ-Glu-Gly, γ-Glu-Ser and γ-Glu-Citrulline excluding methionine suboxide, AST and ALT which are close to 1 in odds ratio, as apparent from Table 4 given later. It is also possible to raise the accuracy by adding at least one of methionine sulfoxide, AST and ALT.

The present invention also provides a liver disease marker for identifying simple steatosis (SS) which is a combination including at least γ-Glu-Taurine, γ-Glu-Ala, γ-Glu-Leu, γ-Glu-Val, γ-Glu-Glu, AST, ALT, γ-Glu-Thr and γ-Glu-Gln, as apparent from Table 4 given later.

The present invention also provides a liver disease marker for identifying non-alcoholic steatohepatitis which is a combination including at least glucosamine, γ-Glu-Ala, γ-Glu-Val, γ-Glu-Gly, γ-Glu-Gln and γ-Glu-Citrulline excluding AST and ALT which are close to 1 in odds ratio, as apparent from Table 4 given later. It is also possible to raise the accuracy by adding AST and/or ALT.

The present invention also provides a method for measuring a liver disease marker, wherein a γ-Glu-X (X represents an amino acid or an amine) peptide in a sample is measured as a liver disease marker.

The present invention also provides an apparatus for measuring a liver disease marker, the apparatus including means for preparing a test sample suitable for analysis from a collected sample and analysis means for measuring a γ-Glu-X (X represents an amino acid or an amine) peptide in the test sample as a liver disease marker.

The present invention also provides a method for assaying a pharmaceutical preparation, the method including the steps of: measuring a concentration of any of the above-mentioned liver disease biomarkers in blood collected before and after administration of the pharmaceutical preparation; and comparing the measurement results between the blood before administration of the above-mentioned pharmaceutical preparation and the blood after administration thereof.

The present invention also provides a method for assaying a pharmaceutical preparation, the method including the steps of: measuring a concentration of any of the above-mentioned liver disease markers in blood collected from a first group consisting of one or more individuals that received the pharmaceutical preparation and blood collected from a second group consisting of one or more individuals that did not receive the pharmaceutical preparation; and comparing the concentrations of the measured liver disease marker between the first group and the second group.

Furthermore, a method for diagnosing a liver disease according to the present invention includes the steps of: collecting blood from one or more individuals to be diagnosed; measuring a concentration of a marker of the present invention in the collected blood by any of the above-mentioned measuring methods; and comparing the concentration of the marker with that in blood from one or more normal individuals.

A method for diagnosing a toxic side effect caused by an electrophile property of a pharmaceutical preparation (oxidative stress generated by administration of the pharmaceutical preparation) according to the present invention includes the steps of: collecting blood from an individual before and after administration of the pharmaceutical preparation; measuring a concentration of a marker of the present invention in the collected blood by any of the above-mentioned measuring methods; and comparing the concentration of the marker with that in blood from one or more normal individuals. Here, the pharmaceutical preparation may be of any type.

In the above-mentioned methods, the step of measuring the concentration of a marker includes both measuring separately each of the blood samples collected from individuals and measuring a pool of blood collected from a plurality of individuals. The step of comparing the measured concentration of the marker also includes both comparing each of the concentrations obtained from the respective measurements one by one and comparing a cumulative total or a mean value of the concentrations obtained from the respective measurements.

A mammal in which a marker may be used to detect oxidative stress in its tissue is not limited and may be any mammal as long as the mammal experiences oxidative stress in its tissue and a marker of the present invention may be measured in its blood. The mammal is preferably a human.

Although the mammal from which blood used for this diagnosing method is collected is not particularly limited, a mammal whose blood contains at least one of the above-mentioned markers is preferred. Rodents such as a mouse and a rat, a human, a monkey, and a dog are more preferred.

### Advantageous Effects of Invention

According to the present invention, a normal person (C) and liver diseases such as drug-induced liver injury (DI), an asymptomatic hepatitis B carrier (AHB), chronic hepatitis B (CHB), hepatitis C virus carriers with persistently normal ALT (CNALT), chronic hepatitis C (CHC), cirrhosis type C (CIR), hepatocellular carcinoma (HCC), nonalcoholic steatohepatitis (NASH), and simple steatosis (SS) can be identified rapidly by measuring the concentrations of γ-Glu-X peptides and the levels of AST and ALT in blood.

### BRIEF DESCRIPTION OF DRAWINGS

FIG. 1 is a diagram schematically showing a mechanism in which ophthalmic acid is biosynthesized in the presence of an electrophile and active oxygen (oxidative stress).

FIG. 2 is a diagram schematically showing a mechanism in which γ-Glu-X peptides are biosynthesized inpatients with various types of liver disorders.

FIG. 3 is a diagram showing the comparison of the LC-MS measurement results of γ-Glu-X (X represents an amino acid or an amine) peptides in the sera from a normal person (C) and a hepatocellular carcinoma patient (HCC).

FIG. 4 is a drawing showing the comparison of the measurement results obtained by LC-MS with regard to γ-Glu-X peptides in the sera of normal persons (C) and in the sera of asymptomatic hepatitis B carriers (AHB).

FIG. 5 is a drawing showing the comparison of the measurement results obtained by LS-MS with regard to γ-Glu-X peptides in the sera of patients with simple steatosis (SS) and in the sera of patients with non-alcoholic steatohepatitis (NASH).

FIG. 6 is a drawing showing the comparison of the measurement results of AST, ALT and γ-Glu-X peptides in the sera of normal persons and in the sera of patients with various types of hepatitis.

FIG. 7 is a flow chart showing one example of procedures for development and assessment of a multiple logistic regression (MLR) model.

FIG. 8 is a drawing showing the accuracy of a screening test of AST, ALT and γ-Glu-X peptides in normal persons.

FIG. 9 is also a drawing showing the accuracy of the screening test for drug-induced liver injury (DI).

FIG. 10 is also a drawing showing the accuracy of the screening test in asymptomatic hepatitis B carriers (AHB).

FIG. 11 is also a drawing showing the accuracy of the screening test for chronic hepatitis B (CHB).

FIG. 12 is also a drawing showing the accuracy of the screening test in hepatitis C virus carriers with persistently normal ALT (CNALT).

FIG. 13 is also a drawing showing the accuracy of the screening test for chronic hepatitis C (CHC).

FIG. 14 is also a drawing showing the accuracy of the screening test for cirrhosis type C (CIR).

FIG. 15 is also a drawing showing the accuracy of the screening test for hepatocellular carcinoma (HCC).

FIG. 16 is also a drawing showing the accuracy of the screening test for simple steatosis (SS).

FIG. 17 is also a drawing showing the accuracy of the screening test for non-alcoholic steatohepatitis (NASH).

FIG. 18 is a drawing showing the comparison of the concentrations of γ-Glu-X peptides in the sera of patients with hepatocellular carcinoma (HCC) and in the sera of patients with gastric cancer (GC).

FIG. 19 covers boxplots of AFP and MLR as well as receiver operating curves (ROC) for distinguishing patients with hepatocellular carcinoma (HCC) from patients with chronic hepatitis C (CHC) and patients with cirrhosis type C (CIR).

FIG. 20 is a drawing showing the quantitative results of γ-Glu-X and γ-Glu-X-Gly in livers of mice on administration of buthionine sulfoximine (BSO) and diethylmaleate (DEM).

FIG. 21 is also a drawing showing the quantitative results of γ-Glu-X and γ-Glu-X-Gly in livers of mice on administration of APAP.

FIG. 22 is also a drawing showing the quantitative results of γ-Glu-X and γ-Glu-X-Gly in the sera of mice on administration of APAP.

FIG. 23 is a drawing showing a part of data on normal persons (C) and patients with cirrhosis type C (CIR), simple steatosis (SS) and non-alcoholic steatohepatitis (NASH).

FIG. 24 is also a drawing showing another part of the data on normal persons (C) and patients with cirrhosis type C (CIR), simple steatosis (SS) and non-alcoholic steatohepatitis (NASH).

FIG. 25 is also a drawing showing still another part of the data on normal persons (C) and patients with cirrhosis type C (CIR), simple steatosis (SS) and non-alcoholic steatohepatitis (NASH).

FIG. 26 is also a drawing showing the remaining part of the data on normal persons (C) and patients with cirrhosis type C (CIR), simple steatosis (SS) and non-alcoholic steatohepatitis (NASH).

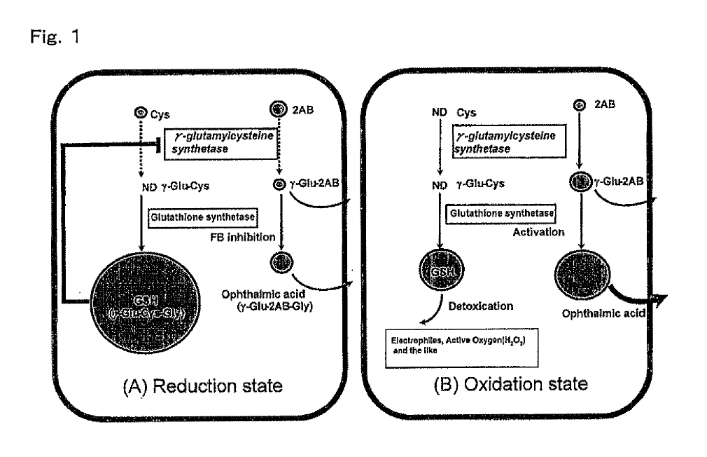

### MODES FOR IMPLEMENTING THE INVENTION

Hereinbelow, the embodiments of the present invention will be described in detail.

As described above, many liver injuries such as hepatitis, hepatic cirrhosis, and hepatocellular carcinoma are known to be closely related to oxidative stress. Thus, concentrations of ophthalmic acid were measured using capillary electrophoresis-time-of-flight mass spectrometry (CE-TOFMS) in the sera from 53 normal persons (C), 10 patients with drug-induced liver injury (DI), 9 asymptomatic hepatitis B carriers (AHB), 7 patients with chronic hepatitis B (CHB), 10 hepatitis type C virus carriers with persistently normal ALT (CNALT), 24 patients with chronic hepatitis C (CHC), 10 patients with cirrhosis type C (CIR), 19 patients with hepatocellular carcinoma (HCC), 11 patients with non-alcoholic steatohepatitis (NASH) and 9 patients with simple steatosis (SS). However, different substances were found to have increased predominantly in the hepatitis patients and all of these substances were identified as γ-Glu-X peptides (note: X represents an amino acid or an amine).

1. Extraction of Metabolites from the Sera

The serum (100 μl) collected from a normal person and patients with various types of hepatitis were added into 900 μl of methanol containing a standard substance to inactivate an enzyme, thereby stopping enhancement of metabolism. After 400 μl of ultrapure water and 1000 μl of chloroform were added, the mixture was centrifuged at 4,600 g for 5 minutes at 4° C. After allowing to stand, 750 μl of separated water-methanol phase was passed through a 5 kDa molecular weight cutoff filter for centrifugal ultrafiltration for deproteinization. The filtrate was lyophilized and 50 μl of Milli-Q water was added thereto. The mixture was subjected to a CE-TOFMS measurement and an LC-MS measurement.

### 2. Measurement of Metabolites in the Sera by Capillary Electrophoresis-Mass Spectroscope (CE-TOFMS)

Low molecular weight metabolic products in the sera from normal persons and hepatitis patients were measured simultaneously using a CE-TOFMS.

### Analysis Conditions for CE-TOFMS

a. Analysis Conditions for Capillary

### Electrophoresis-Electrophoresis (CE)

A fused silica capillary (internal diameter: 50 μm, external diameter: 350 μm, and full length: 100 cm) was used as a capillary. 1M formic acid (pH: approximately 1.8) was used as a buffer solution. Measurement was performed at an applied voltage of +30 kV and at a capillary temperature of 20° C. A sample was injected by pressurization at 50 mbar for 3 seconds (about 3 nl).

b. Analysis Conditions for Time-of-Flight Mass Spectrometer (TOFMS)

Positive ion mode was employed. An ionization voltage, a fragmentor voltage, a skimmer voltage, and an OctRFV voltage were set at 4 kV, 75 V, 50 V, and 125 V, respectively. Nitrogen was used as dry gas, with the temperature set at 300° C. and the pressure set at 10 psig. Fifty percent methanol solution was used as sheath fluid. Reserpine (m/z 609.2807) for mass calibration was mixed into the methanol solution to a final concentration of 0.5 μM and the resultant solution was fed at 10 μl/min. Using the mass numbers of reserpine (m/z 609.2807) and an adduct ion of methanol (m/z 83.0703), all the obtained data were automatically calibrated.

### 3. Measurement of γ-Glu-X Peptides in the Sera by Liquid Chromatography-Mass Spectroscope (LC-MSMS)

To achieve a sensitive measurement, γ-Glu-X peptides in the sera were measured using a LC-MSMS.

a. Analysis Conditions for Liquid Chromatography (LC)

Develosil RPAQUEOUS-AR-3 (2 mm (internal diameter)×100 mm (length), 3 μm) from Nomula Chemical Co. was used as a column for separation, and a column oven was set at 30° C. One microliter of sample was injected into the column. A mobile phase A was 0.5% formic acid and a mobile phase B was acetonitrile. Gamma-Glu-X peptides were separated by an elution method using a gradient of 0% (0 min)-1% (5 min)-10% (15 min)-99% (17 min)-99% (19 min) B solution, at a flow rate of 0.2 ml/min.

b. Analysis Conditions for Triple Quadrupole Mass Spectrometer (QqQMS)

An API3000 triple quadrupole mass spectrometer from Applied Biosystem was used for measurement in MRM mode in positive ion mode. The parameters of the mass spectrometer were shown below:

ionspray voltage: 5.5 kV

nebulizer gas pressure: 12 psi

curtain gas pressure: 8 psi collision gas: 8 unit temperature of nitrogen gas: 550° C.

The MRM parameters optimized for measuring the γ-Glu-X peptides in a MRM (Multiple Reaction Monitoring) mode are shown in Table 1.

TABLE 1

| | | | | | | Outlet |
|---|---|---|---|---|---|---|
| | | | Declustering | Clustering | Collision | voltage of |
| | Q1 | Q2 | voltage | voltage | energy | collision |
| Peptides | (m/z) | (m/z) | (V) | (V) | (V) | cell (V) |
| γ-Glu-Gly | 205.2 | 84.2 | 11 | 60 | 31 | 4 |
| γ-Glu-Ala | 219.0 | 90.3 | 26 | 100 | 17 | 6 |
| γ-Glu-Ser | 235.2 | 106.4 | 36 | 90 | 17 | 18 |
| γ-Glu-Val | 247.0 | 118.0 | 26 | 120 | 17 | 8 |
| γ-Glu-Norvaline | 247.0 | 118.0 | 26 | 120 | 17 | 8 |
| γ-Glu-Thr | 249.2 | 120.0 | 51 | 80 | 17 | 8 |
| γ-Glu-Homoserine | 249.2 | 120.0 | 51 | 80 | 17 | 8 |
| γ-Glu-Taurine | 255.2 | 126.0 | 46 | 170 | 19 | 8 |
| γ-Glu-Ile | 261.0 | 132.0 | 31 | 150 | 17 | 8 |
| γ-Glu-Leu | 261.0 | 132.0 | 31 | 150 | 17 | 8 |
| γ-Glu-Norleucine | 261.0 | 132.0 | 31 | 150 | 17 | 8 |
| γ-Glu-Asn or γ-Glu-Gly-Gly | 262.2 | 133.0 | 16 | 50 | 17 | 22 |
| γ-Glu-Ornithine | 262.3 | 132.9 | 31 | 90 | 17 | 8 |
| γ-Glu-Asp | 263.2 | 134.1 | 31 | 150 | 17 | 8 |
| γ-Glu-Homocysteine | 265.1 | 135.9 | 36 | 140 | 17 | 8 |
| γ-Glu-Gln | 276.2 | 146.9 | 31 | 100 | 17 | 10 |
| γ-Glu-Ala-Gly | 276.2 | 146.9 | 31 | 100 | 17 | 10 |
| γ-Glu-Lys | 276.2 | 146.9 | 31 | 100 | 17 | 10 |
| γ-Glu-Glu | 277.2 | 148.1 | 26 | 150 | 17 | 10 |
| γ-Glu-Met | 279.2 | 149.9 | 36 | 50 | 17 | 6 |
| γ-Glu-His | 285.2 | 155.9 | 21 | 110 | 17 | 8 |
| γ-Glu-Ser-Gly | 292.2 | 163.0 | 26 | 170 | 17 | 14 |
| γ-Glu-Phe | 295.1 | 166.1 | 41 | 220 | 17 | 14 |
| γ-Glu-Val-Gly | 304.2 | 175.0 | 31 | 100 | 17 | 5 |
| γ-Glu-Norvaline-Gly | 304.2 | 175.0 | 31 | 100 | 17 | 5 |
| γ-Glu-Arg | 304.3 | 174.9 | 46 | 110 | 21 | 12 |
| γ-Glu-Citrulline | 305.2 | 158.9 | 23 | 110 | 19 | 8 |
| γ-Glu-Thr-Gly | 306.3 | 177.1 | 31 | 100 | 17 | 10 |
| γ-Glu-Homoserine-Gly | 306.3 | 177.1 | 31 | 100 | 17 | 10 |
| γ-Glu-Tyr | 311.3 | 181.9 | 56 | 70 | 19 | 10 |
| γ-Glu-Ile-Gly | 318.3 | 189.3 | 31 | 100 | 17 | 5 |
| γ-Glu-Leu-Gly | 318.3 | 189.3 | 31 | 100 | 17 | 5 |
| γ-Glu-Asn-Gly | 319.2 | 173.2 | 41 | 170 | 19 | 18 |
| γ-Glu-Asp-Gly | 320.2 | 191.2 | 26 | 130 | 17 | 10 |
| γ-Glu-Homocysteine-Gly | 322.2 | 193.3 | 26 | 150 | 17 | 12 |
| γ-Glu-Gln-Gly | 333.3 | 84.0 | 16 | 70 | 47 | 4 |
| γ-Glu-Glu-Gly | 334.3 | 187.3 | 21 | 90 | 23 | 14 |
| γ-Glu-Trp | 334.2 | 188.1 | 41 | 200 | 23 | 14 |
| γ-Glu-Tyr-Gly | 368.2 | 135.9 | 31 | 180 | 27 | 12 |
| Ophthalmate (γ-Glu-2AB-Gly) | 290.3 | 160.9 | 11 | 80 | 15 | 8 |
| GSSG | 307.3 | 130.0 | 26 | 140 | 19 | 8 |
| GSH (γ-Glu-Cys-Gly) | 308.2 | 179.0 | 36 | 130 | 17 | 10 |

### 4. Search and Evaluation of Liver Injury Biomarkers

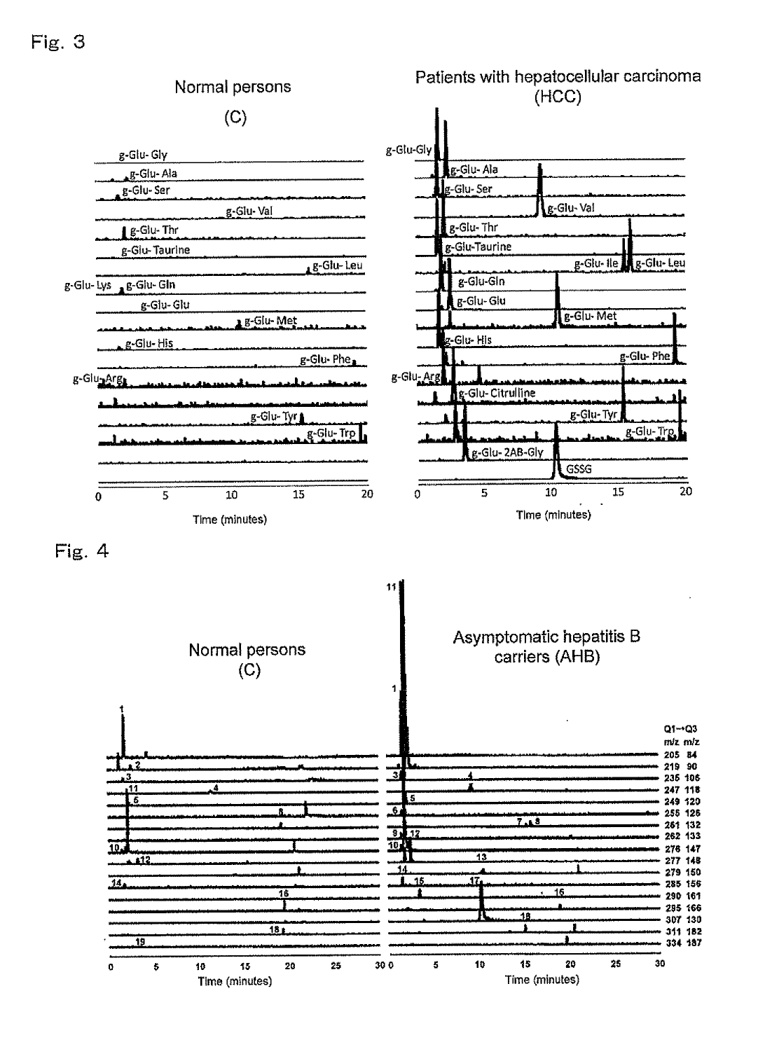

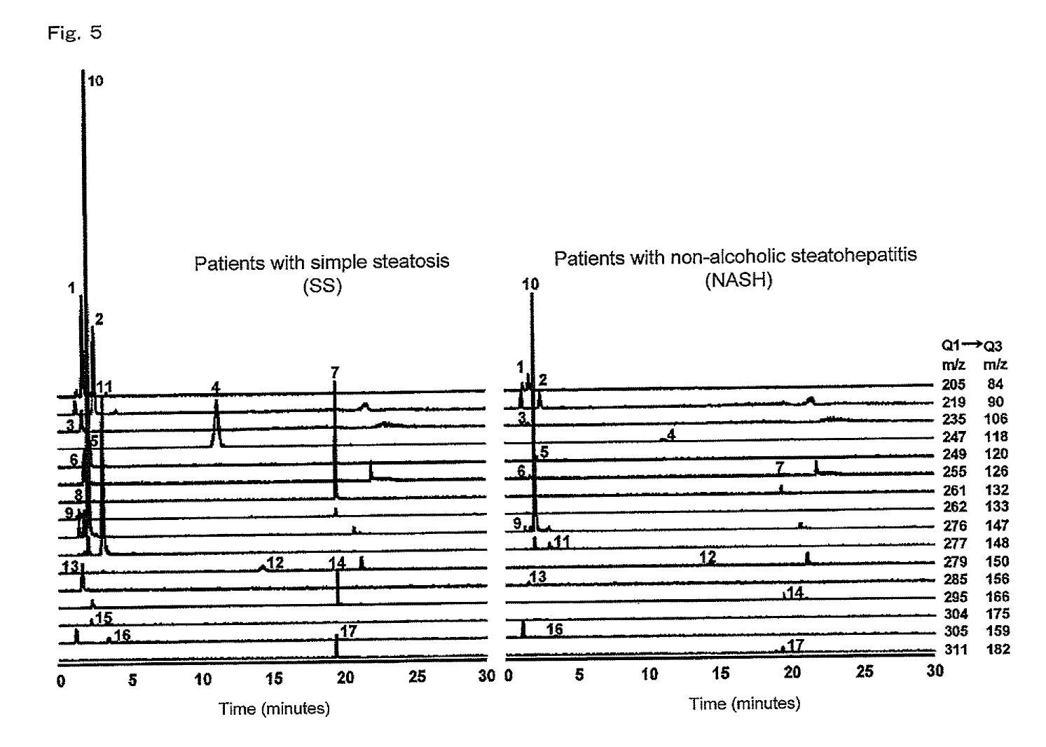

FIG. 3 shows measurement results of γ-Glu-X peptides in the sera obtained from normal persons (C) and patients with hepatocellular carcinoma (HCC) by using LC-MS. FIG. 4 shows measurement results of γ-Glu-X peptides in the sera obtained from normal persons (C) and asymptomatic hepatitis B carriers (AHB) by using LC-MS. FIG. 5 shows measurement results of γ-Glu-X peptides in the sera obtained from patients with simple steatosis (SS) and patients with non-alcoholic steatohepatitis (NASH) by using LC-MS. In FIG. 3 and FIG. 4, 1 represents γ-Glu-Gly; 2 represents γ-Glu-Ala; 3 represents γ-Glu-Ser; 4 represents γ-Glu-Val; 5 represents γ-Glu-Thr; 6 represents γ-Glu-Taurine; 7 represents γ-Glu-Ile; 8 represents γ-Glu-Leu; 9 represents γ-Glu-Asn; 10 represents γ-Glu-Lys; 11 represents γ-Glu-Gln; 12 represents γ-Glu-Glu; 13 represents γ-Glu-Met; 14 represents γ-Glu-His; 15 represents ophthalmate (γ-Glu-2AB-Gly); 16 represents γ-Glu-Phe; 17 represents oxidized-type glutathione (GSSG); 18 represents γ-Glu-Tyr; and 19 represents γ-Glu-Glu-Gly. It was found that many γ-Glu-X peptides are increased in patients with hepatocellular carcinoma (HCC) and asymptomatic hepatitis B carriers (AHB), as compared with a normal person (C) and differences between patients with simple steatosis (SS) and patients with non-alcoholic steatohepatitis (NASH). The concentrations of the γ-Glu-X peptides were also significantly higher in patients with other liver injuries than in normal persons.

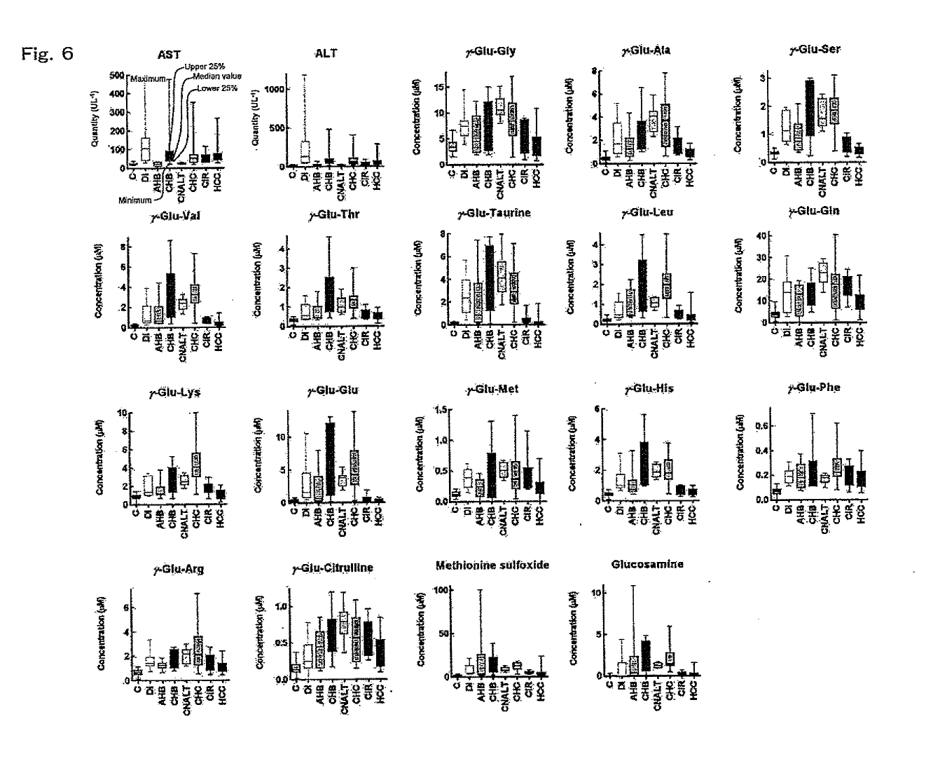

FIG. 6 shows the measurement results of an AST level, an ALT level, and γ-Glu-X peptides in the sera from a normal person (C) and patients with various types of hepatitis. In this figure, the arrows show the maximum and the minimum, the top of the box corresponds to a measurement ranked in the top 25% and the bottom of the box corresponds to a measurement ranked in the bottom 75%, and the transverse line in the box shows a median.

Although the AST level and the ALT level, which are conventional liver function test values, had increased in drug-induced liver injury (DI), chronic hepatitis B (CHB), and chronic hepatitis C (CHC), their levels in the other types of hepatitis were not significantly different from the levels in the normal person.

However, patients with drug-induced liver injury (DI) showed a higher level of γ-Glu-X peptides such as γ-Glu-Ser and γ-Glu-Thr than the normal person (C), and patients with the other types of hepatitis showed an even higher level of γ-Glu-X peptides. Particularly, patients with asymptomatic hepatitis B (AHB), asymptomatic hepatitis C (AHC), or hepatocellular carcinoma (HCC) also showed a high level of γ-Glu-X peptides. Furthermore, it was observed in more detailed investigation that some γ-Glu-X peptides were found at a higher level in asymptomatic hepatitis C (AHC) than in asymptomatic hepatitis B (AHB), and in chronic hepatitis B (CHB) than in asymptomatic hepatitis B (AHC), and some γ-Glu-X peptides showed a tendency to decrease as hepatitis C progressed from asymptomatic hepatitis C (AHC) to chronic hepatitis C (CHC), and further to hepatocellular carcinoma (HCC) even if all of them are derived from the same hepatitis C.

As described above, the blood concentrations of AST, AZT, and each of the γ-Glu-X peptides were different in each of the diseases. Therefore, we expected that the diseases could be classified by using the measurements of these components. Then, multiple logistic regression analysis, which is a methodology for multivariate analysis, was performed to select a biomarker for distinguishing each liver disease. The result is shown in Tables 2-4.

TABLE 2

| | | | | 95% CI | | | 95% CI | | |
|---|---|---|---|---|---|---|---|---|---|
| | | | Standard | Lower | Upper | Odds | Lower | Upper | |
| Group | Marker | Parameter | deviation | limit | limit | ratio | limit | limit | p-value |
| C | (Intercept) | 3.29 × 10² | 1.06 × 10⁵ | — | — | — | — | — | 0.9975 |
| | Glucosamine | 64.1 | 6.22 × 10⁴ | — | — | 6.58 × 10²⁷ | 0.00 | 0.00 | 0.9992 |
| | γ-Glu-Ala | 1.18 | 1.29 × 10⁵ | — | — | 3.25 | 0.00 | 0.00 | 1 |
| | Methionine | −58.5 | 1.89 × 10⁴ | — | — | 3.92 × 10⁻²⁶ | 0.00 | 0.00 | 0.9975 |
| | sulfoxide | | | | | | | | |
| | γ-Glu-Leu | −1.48 × 10² | 3.87 × 10⁵ | — | — | 0.00 | 0.00 | 0.00 | 0.9997 |
| | γ-Glu-Val | 1.31 × 10² | 3.01 × 10⁵ | — | — | 1.14 × 10⁻⁷ | 0.00 | 0.00 | 0.9997 |
| | AST | −2.37 | 2.19 × 10³ | — | — | 0.0933 | 0.00 | 0.00 | 0.9991 |
| | ALT | −3.30 | 3.64 × 10³ | — | — | 0.0370 | 0.00 | 0.00 | 0.9993 |
| | γ-Glu-Phe | −3.05 × 10² | 1.18 × 10⁶ | — | — | 0.00 | 0.00 | 0.00 | 0.9998 |
| | γ-Glu-Met | 3.04 × 10² | 6.70 × 10⁵ | — | — | 8.00 × 10¹³¹ | 0.00 | 0.00 | 0.9996 |
| | γ-Glu-Gln | −14.5 | 6.59 × 10³ | — | — | 5.03 × 10⁻⁷ | 0.00 | 0.00 | 0.9982 |
| DI | (Intercept) | −4.12 | 1.43 | −7.38 | −1.68 | — | — | — | 0.004 |
| | γ-Glu-Taurine | 2.34 | 1.06 | 0.533 | 4.66 | 10.4 | 1.70 | 1.06 × 10² | 0.027 |
| | γ-Glu-Leu | −17.9 | 5.56 | −31.3 | −9.13 | 1.64 × 10⁻⁸ | 2.66 × 10⁻¹⁴ | 1.08 × 10⁻⁴ | 0.0013 |
| | γ-Glu-Glu | 0.322 | 0.316 | −0.270 | 1.07 | 1.38 | 0.763 | 2.93 | 0.3081 |
| | AST | −0.0346 | 0.0125 | −0.0633 | −0.0133 | 0.966 | 0.939 | 0.987 | 0.0058 |
| | ALT | 0.0521 | 0.0194 | 0.0200 | 0.0967 | 1.05 | 1.02 | 1.10 | 0.0073 |
| | γ-Glu-Gly | 0.110 | 0.271 | −0.430 | 0.679 | 1.12 | 0.650 | 1.97 | 0.6857 |
| | γ-Glu-Arg | 3.57 | 1.43 | 1.07 | 6.73 | 35.6 | 2.92 | 8.41 × 10² | 0.0125 |
| | γ-Glu-Ser | 1.25 | 1.79 | −4.26 | 4.57 | 3.50 | 0.0142 | 96.7 | 0.4851 |
| | γ-Glu-Phe | 7.94 | 11.2 | −15.2 | 29.5 | 2.80 × 10³ | 2.61 × 10⁻⁷ | 6.26 × 10¹² | 0.4787 |
| | γ-Glu-Met | 10.9 | 8.03 | −3.48 | 27.9 | 5.21 × 10⁴ | 0.0309 | 1.25 × 10¹² | 0.1764 |
| | γ-Glu-Citrulline | −6.21 | 4.04 | −15.2 | 0.977 | 2.01 × 10⁻³ | 2.60 × 10⁻⁷ | 2.66 | 0.1241 |
| AHB | (Intercept) | 2.07 | 1.05 | 0.142 | 4.28 | — | — | — | 0.0477 |
| | γ-Glu-Taurine | 2.99 | 0.825 | 1.68 | 4.91 | 19.8 | 5.38 | 1.35 × 10² | 0.0003 |
| | γ-Glu-Ala | −2.38 | 1.08 | −4.68 | −0.343 | 0.0928 | 9.28 × 10⁻³ | 0.709 | 0.0274 |
| | γ-Glu-Leu | 12.9 | 3.38 | 7.20 | 20.6 | 4.11 × 10⁵ | 1.34 × 10³ | 8.51 × 10⁸ | 0.0001 |
| | γ-Glu-Val | −7.99 | 2.50 | −13.7 | −3.83 | 3.40 × 10⁻⁴ | 1.13 × 10⁻⁶ | 0.0216 | 0.0014 |
| | AST | −0.267 | 0.0683 | −0.410 | −0.141 | 0.766 | 0.664 | 0.868 | <.0001 |
| | ALT | 0.0800 | 0.0244 | 0.0345 | 0.130 | 1.08 | 1.04 | 1.14 | 0.001 |
| | γ-Glu-Lys | −3.13 | 0.897 | −5.11 | −1.54 | 0.0435 | 6.05 × 10⁻³ | 0.215 | 0.0005 |
| | γ-Glu-Arg | 1.23 | 1.20 | −0.914 | 3.82 | 3.42 | 0.401 | 45.5 | 0.3071 |
| | γ-Glu-Met | 3.07 | 5.49 | −8.89 | 12.8 | 21.5 | 1.37 × 10⁻⁴ | 3.60 × 10⁵ | 0.5765 |
| | γ-Glu-Gln | 0.422 | 0.105 | 0.234 | 0.652 | 1.52 | 1.26 | 1.92 | <.0001 |

TABLE 3

| | | | | 95% CI | | | 95% CI | | |
|---|---|---|---|---|---|---|---|---|---|
| | | | Standard | Lower | Upper | Odds | Lower | Upper | |
| Group | Marker | Parameter | deviation | limit | limit | ratio | limit | limit | p-value |
| CHB | (Intercept) | −1.58 × 10² | 5.38 × 10⁴ | — | — | — | — | — | 0.9977 |
| | γ-Glu-Ala | −1.62 × 10² | 5.15 × 10⁴ | — | — | 0.00 | 0.00 | 0.00 | 0.9975 |
| | Methionine | 39.3 | 1.64 × 10⁴ | 5.41 | — | 1.22 × 10¹⁷ | 2.24 × 10² | 0.00 | 0.9981 |
| | sulfoxide | | | | | | | | |
| | γ-Glu-Leu | −60.0 | 9.98 × 10⁴ | — | — | 8.86 × 10⁻²⁷ | 0.00 | 0.00 | 0.9995 |
| | γ-Glu-Glu | −82.9 | 3.17 × 10⁴ | — | — | 9.90 × 10⁻³⁷ | 0.00 | 0.00 | 9.98E−01 |
| | AST | −1.38 | 9.14 × 10² | — | — | 0.252 | 0.00 | 0.00 | 0.9988 |
| | ALT | 2.81 | 1.10 × 10³ | — | — | 16.6 | 0.00 | 0.00 | 0.998 |
| | γ-Glu-Arg | −97.6 | 6.28 × 10⁴ | — | — | 4.01 × 10⁻⁴³ | 0.00 | 0.00 | 0.9988 |
| | γ-Glu-Ser | −49.7 | 8.07 × 10⁴ | — | — | 2.48 × 10⁻²² | 0.00 | 0.00 | 0.9995 |
| | γ-Glu-His | 3.25 × 10² | 1.01 × 10⁵ | — | — | 1.90 × 10¹⁴¹ | 0.00 | 0.00 | 0.9974 |
| | γ-Glu-Phe | −2.09 × 10³ | 6.60 × 10⁶ | — | — | 0.00 | 0.00 | 0.00 | 0.9975 |
| | γ-Glu-Met | 1.35 × 10² | 1.60 × 10⁵ | — | — | 3.84 × 10⁵⁸ | 0.00 | 0.00 | 0.9993 |
| | γ-Glu-Citrulline | 7.65 × 10² | 2.72 × 10⁵ | — | — | — | 0.00 | 0.00 | 0.9978 |
| CNALT | (Intercept) | −65.4 | 2.92 × 10⁴ | 5.67 × 10⁴ | — | — | — | — | 0.9982 |
| | Glucosamine | 21.9 | 3.85 × 10⁴ | — | — | 3.35 × 10⁹ | 0.00 | 0.00 | 0.9995 |
| | γ-Glu-Leu | −3.37 × 10² | 9.98 × 10⁴ | — | — | 0.00 | 0.00 | 0.00 | 0.9973 |
| | γ-Glu-Val | 1.37 × 10² | 4.66 × 10⁴ | — | — | 5.10 × 10⁵⁹ | 0.00 | 0.00 | 0.9976 |
| | AST | −0.255 | 1.45 × 10³ | — | — | 0.775 | 0.00 | 0.00 | 0.9999 |
| | ALT | −0.0435 | 7.74 × 10² | — | — | 0.957 | 0.00 | 0.00 | 1 |
| | γ-Glu-Gly | 8.89 | 5.24 × 10³ | — | — | 7.28 × 10³ | 0.00 | 0.00 | 0.9986 |
| | γ-Glu-Gln | 1.69 | 1.47 × 10³ | — | — | 5.43 | 0.00 | 0.00 | 0.9991 |
| | γ-Glu-Citrulline | 25.5 | 2.86 × 10⁴ | — | — | 1.24 × 10¹¹ | 0.00 | 0.00 | 0.9993 |
| CHC | (Intercept) | −1.83 | 0.431 | −2.73 | −1.03 | — | — | — | <.0001 |
| | Glucosamine | 1.40 | 0.496 | 0.542 | 2.49 | 4.05 | 1.72 | 12.1 | 0.0048 |
| | Methionine | −0.0106 | 0.0503 | −0.112 | 0.0903 | 0.989 | 0.894 | 1.09 | 0.8337 |
| | sulfoxide | | | | | | | | |
| | ALT | −6.21 × 10⁻³ | 3.39 × 10⁻³ | −0.0146 | −1.28 × 10⁻⁴ | 0.994 | 0.986 | 1.000 | 0.0671 |
| | γ-Glu-Lys | 1.22 | 0.255 | 0.765 | 1.78 | 3.38 | 2.15 | 5.94 | <.0001 |
| | γ-Glu-His | −1.82 | 0.547 | −3.05 | −0.836 | 0.163 | 0.0474 | 0.433 | 0.0009 |

TABLE 4

| | | | | 95% CI | | | 95% CI | | |
|---|---|---|---|---|---|---|---|---|---|
| | | | Standard | Lower | Upper | Odds | Lower | Upper | |
| Group | Marker | Parameter | deviation | limit | limit | ratio | limit | limit | p-value |
| CIR | (Intercept) | −23.9 | 7.53 | −49.0 | −12.1 | — | — | — | 0.0015 |
| | Glucosamine | −36.1 | 12.9 | −84.3 | −17.2 | 2.05 × 10⁻¹⁶ | 2.42 × 10⁻³⁷ | 3.56 × 10⁻⁸ | 0.0051 |
| | Methionine | 3.46 | 1.18 | 1.69 | 7.59 | 31.9 | 5.42 | 1.97 × 10³ | 0.0033 |
| | sulfoxide | | | | | | | | |
| | γ-Glu-Leu | 3.30 | 12.3 | — | 26.0 | 27.1 | 0.00 | 1.93 × 10¹¹ | 0.7888 |
| | γ-Glu-Val | −1.57 | 8.20 | −17.0 | 20.4 | 0.208 | 4.28 × 10⁻⁸ | 7.59 × 10⁸ | 0.8482 |
| | γ-Glu-Glu | −9.77 | 3.43 | −19.3 | −4.11 | 5.74 × 10⁻⁵ | 4.15 × 10⁻⁹ | 0.0163 | 0.0044 |
| | AST | 0.0123 | 0.0296 | — | — | 1.01 | 0.00 | 0.00 | 0.6773 |
| | ALT | −0.0264 | 0.0489 | −0.130 | — | 0.974 | 0.878 | 0.00 | 0.5888 |
| | γ-Glu-Gly | 0.692 | 0.362 | 0.0356 | 1.81 | 2.00 | 1.04 | 6.13 | 0.0557 |
| | γ-Glu-Met | 18.6 | 9.17 | 4.89 | 42.3 | 1.18 × 10⁸ | 1.33 × 10² | 2.45 × 10¹⁸ | 0.0427 |
| | γ-Glu-Gln | 1.04 | 0.372 | 0.435 | 2.06 | 2.84 | 1.54 | 7.84 | 0.0051 |
| | γ-Glu-Citrulline | 1.17 | 7.40 | −12.6 | 26.5 | 3.23 | 3.38 × 10⁻⁶ | 3.14 × 10¹¹ | 0.874 |
| HCC | (Intercept) | −1.55 | 0.771 | −3.13 | 0.0724 | — | — | — | 0.0437 |
| | γ-Glu-Taurine | −0.547 | 0.745 | −2.09 | 0.948 | 0.578 | 0.124 | 2.58 | 0.4628 |
| | Methionine | 0.163 | 0.0578 | 0.0594 | 0.287 | 1.18 | 1.06 | 1.33 | 0.0049 |
| | sulfoxide | | | | | | | | |
| | γ-Glu-Glu | −1.57 | 2.00 | −5.81 | 2.02 | 0.208 | 2.99 × 10⁻³ | 7.53 | 0.4311 |
| | AST | 0.114 | 0.0284 | 0.0628 | 0.175 | 1.12 | 1.06 | 1.19 | <.0001 |
| | ALT | −0.0449 | 0.0174 | −0.0850 | −0.0124 | 0.956 | 0.919 | 0.988 | 0.0099 |
| | γ-Glu-Gly | 0.242 | 0.144 | −0.0165 | 0.558 | 1.27 | 0.984 | 1.75 | 0.0918 |
| | γ-Glu-Ser | −11.7 | 3.73 | −19.9 | −4.95 | 7.96 × 10⁻⁶ | 2.35 × 10⁻⁹ | 7.10 × 10⁻³ | 0.0016 |
| | γ-Glu-Citrulline | 7.83 | 2.07 | 4.05 | 12.3 | 2.51 × 10³ | 57.1 | 2.16 × 10⁵ | 0.0002 |
| SS | (Intercept) | 9.85 | 3.31 × 10⁴ | 4.99 × 10⁵ | — | — | — | — | 0.9998 |
| | γ-Glu-Taurine | −8.83 | 7.22 × 10³ | — | — | 1.46 × 10⁻⁴ | 0.00 | 0.00 | 0.999 |
| | γ-Glu-Ala | −41.1 | 1.85 × 10⁴ | — | 3.97 × 10⁵ | 1.48 × 10⁻¹⁸ | 0.00 | — | 0.9982 |
| | γ-Glu-Leu | −75.9 | 5.90 × 10⁴ | 5.35 × 10³ | — | 1.12 × 10⁻³³ | — | 0.00 | 0.999 |
| | γ-Glu-Val | 19.9 | 2.69 × 10⁴ | 1.85 × 10³ | — | 4.35 × 10⁸ | — | 0.00 | 0.9994 |
| | γ-Glu-Glu | 28.6 | 1.00 × 10⁴ | — | — | 2.61 × 10¹² | 0.00 | 0.00 | 0.9977 |
| | AST | 0.402 | 4.39 × 10² | — | 4.02 × 10² | 1.49 | 0.00 | 4.70 × 10¹⁷⁴ | 0.9993 |
| | ALT | −0.144 | 1.85 × 10² | — | 3.27 × 10² | 0.866 | 0.00 | 1.30 × 10¹⁴² | 0.9994 |
| | γ-Glu-Thr | 28.9 | 5.24 × 10⁴ | — | 4.76 × 10⁵ | 3.65 × 10¹² | 0.00 | — | 0.9996 |
| | γ-Glu-Gln | 8.51 | 4.50 × 10³ | — | 4.13 × 10⁴ | 4.94 × 10³ | 0.00 | — | 0.9985 |
| NASH | (Intercept) | −0.420 | 0.807 | −2.07 | 1.21 | — | — | — | 0.6033 |
| | Glucosamine | 3.89 | 0.762 | 2.53 | 5.56 | 48.8 | 12.5 | 2.60 × 10² | <.0001 |
| | γ-Glu-Ala | 2.70 | 0.678 | 1.57 | 4.30 | 14.9 | 4.83 | 73.7 | <.0001 |
| | γ-Glu-Val | −2.92 | 0.630 | −4.34 | −1.82 | 0.0541 | 0.0130 | 0.162 | <0.001 |
| | AST | −0.0521 | 0.0162 | −0.0913 | −0.0245 | 0.949 | 0.913 | 0.976 | 0.0013 |
| | ALT | 0.0388 | 0.0122 | 0.0166 | 0.0656 | 1.04 | 1.02 | 1.07 | 0.0015 |
| | γ-Glu-Gly | −0.634 | 0.197 | −1.07 | −0.283 | 0.531 | 0.344 | 0.754 | 0.0013 |
| | γ-Glu-Gln | −0.164 | 0.0960 | −0.380 | 8.33 × 10⁻³ | 0.849 | 0.684 | 1.01 | 0.0873 |
| | γ-Glu-Citrulline | −1.84 | 1.71 | −5.31 | 1.54 | 0.158 | 4.93 × 10⁻³ | 4.65 | 0.2812 |

In multiple logistic regression (MLR) analysis, a regression equation (1) for p is solved:

ln(p/1−p)=b₀+b₁x₁+b₂x₂+b₃x₃+ . . . +bₖxₖ (1),

wherein explanatory variables of the number k, such as x₁, x₂, x₃, . . . , xₖ, are used for calculating ratio p, which is an objective variable. Values of the parameters in Table 2 are specific values for b₀, b₁, . . . bₖ in the equation (1). The (Intercept) represents the value of a constant term (b₀).

To calculate probabilities for each case, for example in a group of patients with drug-induced liver injury (DI), in the Table, a value of (Intercept) of −4.12 is given as b₀; a value of γ-Glu-Taurine of 2.34, b₁; a value of γ-Glu-Leu of −17.9, b₂; a value of γ-Glu-Glu of 0.322, b₃; a value of AST of −0.0346, b₄; a value of ALT of 0.0521, b₅; a value of 0.110, b₆; a value of γ-Glu-Arg of 3.57, b₇; a value of γ-Glu-Ser of 1.25, b₈; a value of γ-Glu-Phe of 7.94, b₉; a value of γ-Glu-Met of 10.9, b₁₀; and a value of γ-Glu-Citrulline of −6.21, b₁₁. And, a quantitative value of γ-Glu-Taurine is substituted for x₁; a quantitative value of γ-Glu-Leu, x₂; a quantitative value of γ-Glu-Glu, x₃; a quantitative value of AST, x₄; a quantitative value of ALT, x₅; a quantitative value of γ-Glu-Gly, x₆; a quantitative value of γ-Glu-Arg, x₇; a quantitative value of γ-Glu-Ser, x₈; a quantitative value of γ-Glu-Phe, x₈; a quantitative value of γ-Glu-Met, x₁₀; and a quantitative value of γ-Glu-Citrulline, x₁₁, thereby providing specific values. Estimated standard errors and 95% confidence intervals of the parameters are also indicated in the table.

Based on the result of this analysis, candidate biomarkers were discovered that allowed for a selective distinguishment of many types of hepatitis patients as well as normal persons (C). Biomarkers for identifying a normal person (C) are, for example, glucosamine, γ-Glu-Ala, methionine sulfoxide, γ-Glu-Leu, γ-Glu-Val, AST, ALT, γ-Glu-Phe, γ-Glu-Met and γ-Glu-Gln. It is found that the values thereof can be used to identifying a normal person from other patients with liver diseases. Among these biomarkers, a value of γ-Glu-Met which is greatest exceeding 1 in odds ratio which expresses the extent of variation of probability p when a quantitative value x₁ is increased by 1 makes the greatest contribution to determination of a normal person (C). On the other hand, γ-Glu-Leu and γ-Glu-Phe, each odds ratio of which is zero, contribute to determination of those other than the a normal person (non C) when these substances are increased. Furthermore, FIG. 6 indicates that γ-Glu-Ser, γ-Glu-Thr, γ-Glu-Gly, and γ-Glu-Glu can also be used.

Further, biomarkers of drug-induced liver injury (DI) are γ-Glu-Taurine, γ-Glu-Leu, γ-Glu-Glu, AST, ALT, γ-Glu-Gly, γ-Glu-Arg, γ-Glu-Ser, γ-Glu-Phe, γ-Glu-Met and γ-Glu-Citrulline. These markers are combined to perform, for example, a multiple logistic regression (MLR) analysis. And, when a p-value obtained from Mann-Whitney tests is greater than a predetermined value, for example, 0.5, it is possible to distinguish patients with drug-induced liver injury from patients with other liver diseases. Among these biomarkers, a value of γ-Glu-Met which is greatest exceeding 1 in odds ratio which expresses the extent of variation of a probability p when a quantitative value x₁ is increased by 1 has made the greatest contribution to determination of drug-induced liver injury (DI). On the other hand, γ-Glu-Citrulline, the odds ratio of which is close to 0, contributes to determination of normal persons (C) when this substance is increased. AST and ALT are respectively 0.96599 and 1.05346 in odds ratio which are close to 1.0 at which no change in p is found with an increase in variable. Therefore, their contributions are small and may be omitted.

Further, biomarkers of hepatocellular carcinoma (HCC) are γ-Glu-Taurine, methionine sulfoxide, γ-Glu-Glu, AST, ALT, γ-Glu-Gly, γ-Glu-Ser and γ-Glu-Citrulline. They are combined to perform, for example, a multiple logistic regression (MLR) analysis, and when a p-value is greater than a predetermined value, for example, 0.5, it is possible to distinguish patients with hepatocellular carcinoma from patients with other liver diseases. Among these biomarkers, a value of γ-Glu-Citrulline which is greatest exceeding 1 in odds ratio makes the greatest contribution to determination of hepatocellular carcinoma (HCC). On the other hand, γ-Glu-Glu, the odds ratio of which is close to 0, contributes to determination of a liver disease other than hepatocellular carcinoma (non-HCC) when this substance is increased. Still further, methionine sulfoxide, AST and ALT, each odds ratio of which is close to 1, may be omitted.

Similarly, for the other diseases, their respective candidate biomarkers were discovered as shown in Table 2 to Table 4.

Biomarkers of asymptomatic hepatitis B carriers (AHB) are γ-Glu-Taurine, γ-Glu-Ala, γ-Glu-Leu, γ-Glu-Val, AST, ALT, γ-Glu-Lys, γ-Glu-Arg, γ-Glu-Met and γ-Glu-Gln.

Biomarkers of chronic hepatitis B (CHB) are γ-Glu-Ala, methionine sulfoxide, γ-Glu-Leu, γ-Glu-Glu, AST, ALT, γ-Glu-Arg, γ-Glu-Ser, γ-Glu-His, γ-Glu-Phe, γ-Glu-Met and γ-Glu-Citrulline.

Biomarkers of hepatitis type C virus carriers with persistently normal ALT (CNALT) are glucosamine, γ-Glu-Leu, γ-Glu-Val, AST, ALT, γ-Glu-Gly, γ-Glu-Gln and γ-Glu-Citrulline.

Biomarkers of chronic hepatitis C (CHC) are glucosamine, methionine sulfoxide, ALT, γ-Glu-Lys and γ-Glu-His.

Biomarkers of cirrhosis type C (CIR) are glucosamine, methionine sulfoxide, γ-Glu-Leu, γ-Glu-Val, γ-Glu-Glu, AST, ALT, γ-Glu-Gly, γ-Glu-Met, γ-Glu-Gln, and γ-Glu-Citrulline.

Biomarkers of simple steatosis (SS) are γ-Glu-Taurine, γ-Glu-Ala, γ-Glu-Leu, γ-Glu-Val, γ-Glu-Glu, AST, ALT, γ-Glu-Thr and γ-Glu-Gln.

Biomarkers of non-alcoholic steatohepatitis (NASH) are glucosamine, γ-Glu-Ala, γ-Glu-Val, AST, ALT, γ-Glu-Gly, γ-Glu-Gln and γ-Glu-Citrulline.

Of these biomarkers, those, each odds ratio of which is close to 1, may be omitted, for example, ALT (1.083254) on determination of asymptomatic hepatitis B carriers (AHB); ALT (0.957388) on determination of hepatitis type C virus carriers with persistently normal ALT (CNALT); methionine sulfoxide (0.989493) and ALT (0.993806) on determination of chronic hepatitis C (CHC); AST (1.012402) and ALT (0.973926) on determination of cirrhosis type C (CIR); and AST (0.949237) and ALT (1.039583) on determination of non-alcoholic steatohepatitis (NASH), etc.

Regarding the contribution ratios of these biomarkers, the accuracy of the logistic model may also be further increased by adding the data of cases and allowing the model to restudy the data, and modifying the combination of the biomarkers for each identification and the coefficients of the MLR model.

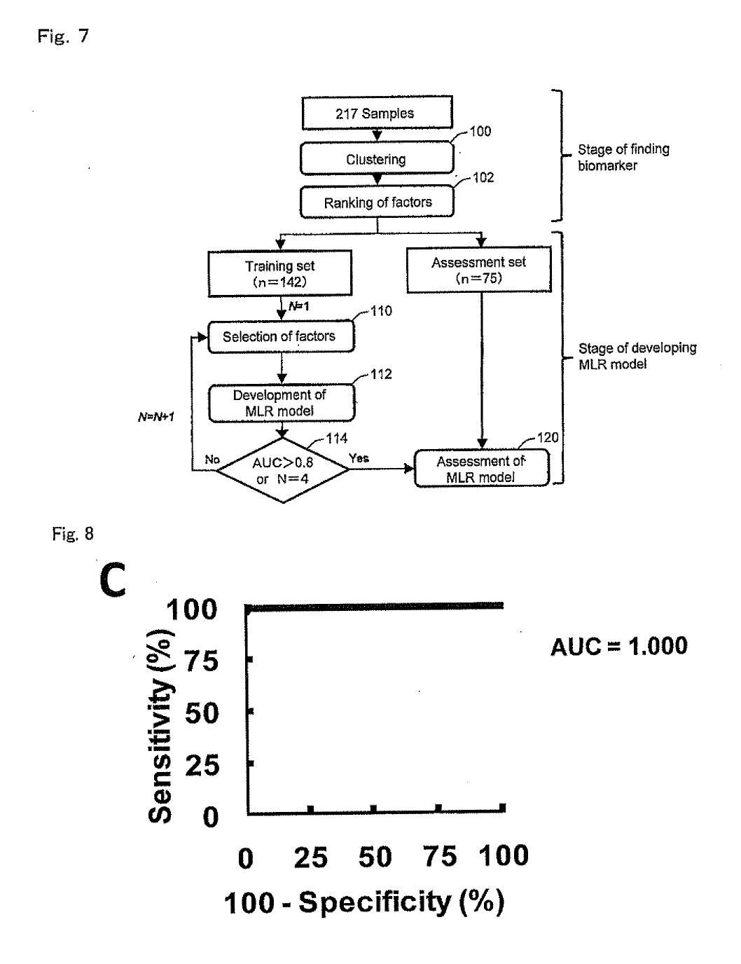

FIG. 7 shows an example of procedures for development and assessment of an MLR model. Ata stage of finding biomarkers, 217 samples were subjected to clustering (Step 100), thereby selecting factors (γ-glutamyl dipeptidase, metabolites and transaminase) which presented a significant change. According to importance for distinguishing samples obtained from sick persons or normal persons from all the other groups, the selected factors were ranked for importance by a method of support vector machine-factor selection (SVM-FS) (Step 102).

At a stage of model development, the MLR model was developed by using factors with importance of the first ranking to the N^{th} ranking, for example, 142 pieces of training data were used to determine a coefficient and a constant term of the formula (I) (Steps 110, 112). Where AUC (area under the receiver-operating curve (ROC)) which is a predictive accuracy of MLR is greater than 0.8 or N becomes 4 (determination results of Step 114: Yes), the model was selected as a final predictor variable.

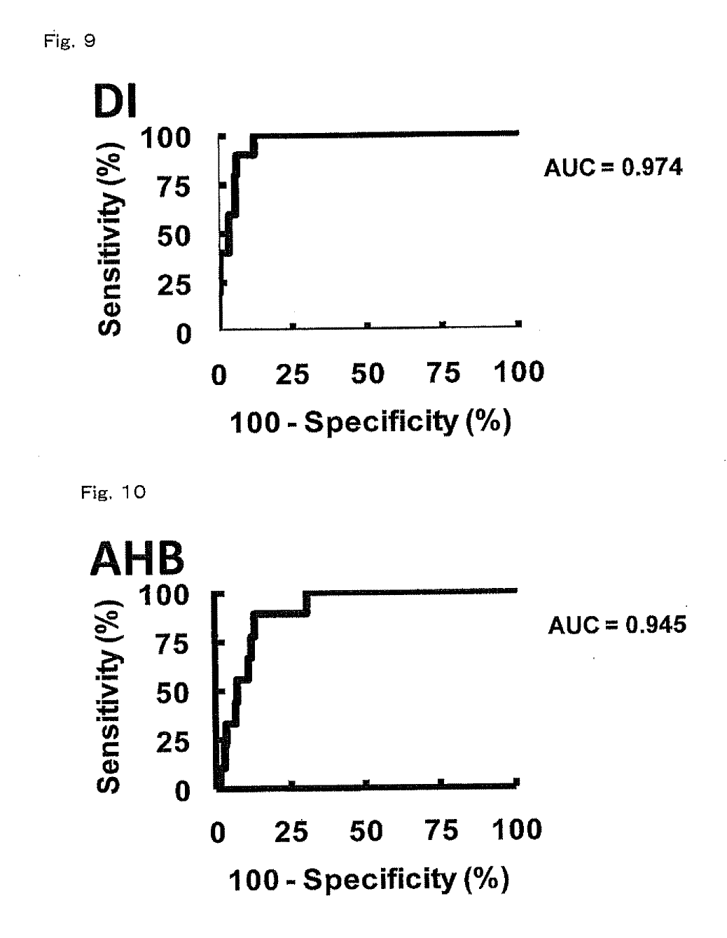

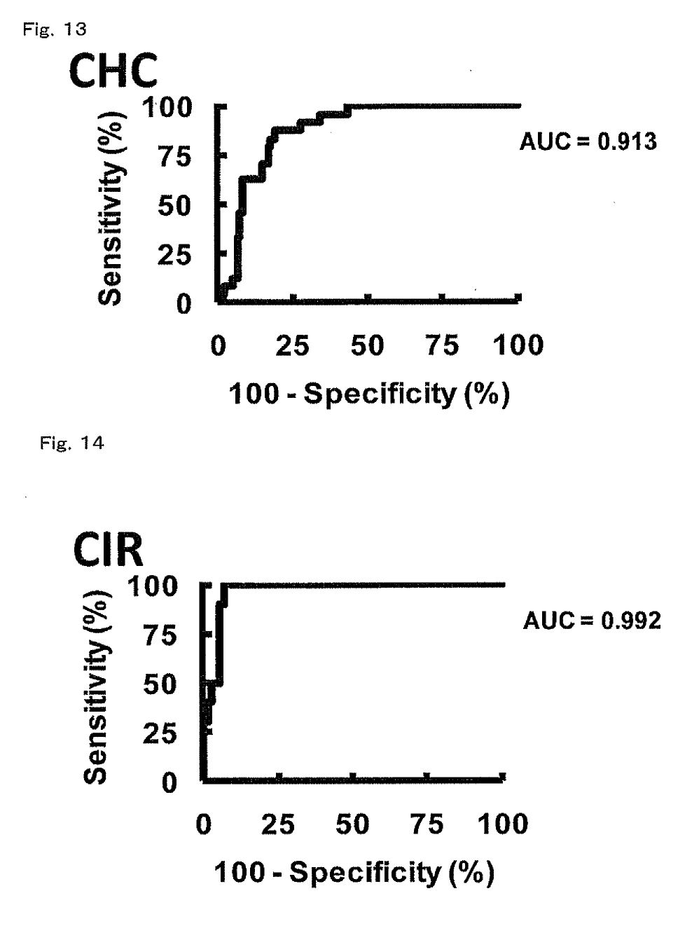

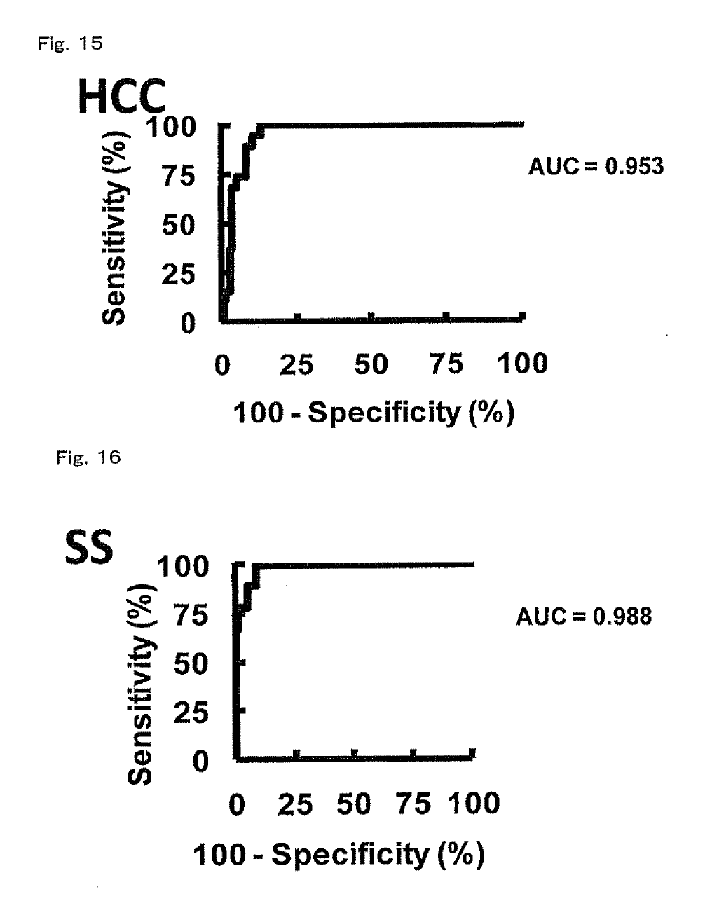

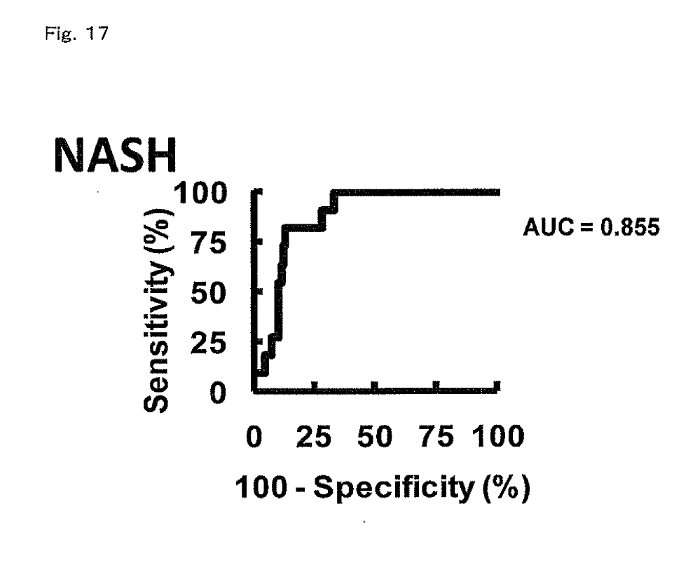

Then, the predictive accuracy of the MLR model was assessed by using, for example, 75 pieces of assessment data (Step 120). FIG. 8 to FIG. 17 show the accuracy of the training data and assessment data predicted by the MLR model, that is, receiver operation curves (ROC) and values of AUC.

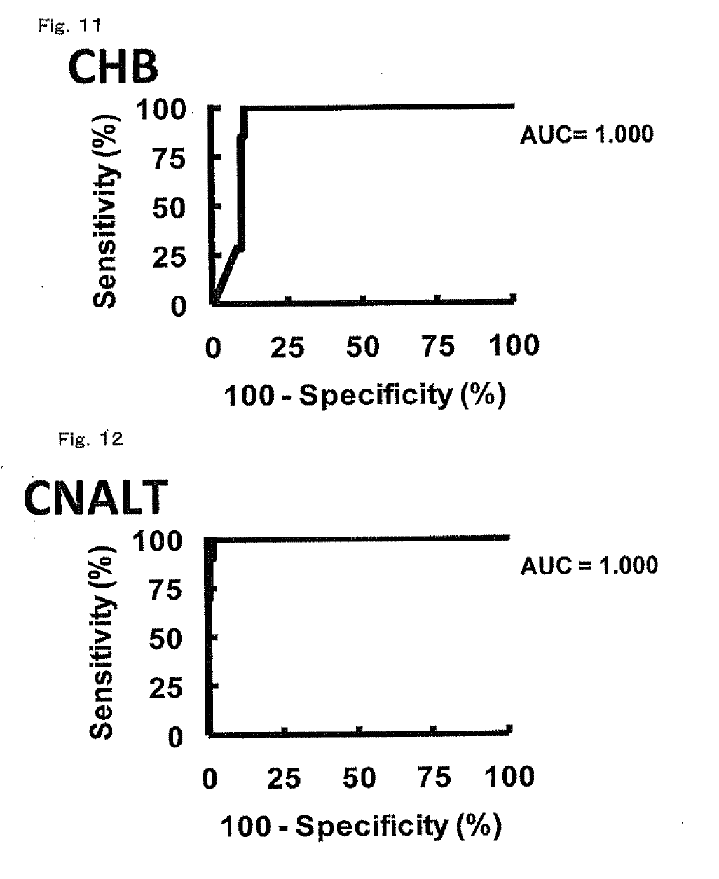

The accuracy of the multiple logistic regression (MLR) model for distinguishing a group of certain patients from all the other groups of patients is shown in FIG. 8 to FIG. 17. For example, in the case of drug-induced liver injury (DI), DI is distinguished from all liver diseases other than DI. As shown in FIG. 8 to FIG. 17, in all diseases, AUC (area under the receiver-operating curve (ROC)) is 0.855 to 1.000. Thus, it was confirmed that various types of liver diseases can be identified at a high accuracy by screening tests for each liver disease with the biomarkers. In particular, normal persons (C) shown in FIG. 8, patients with chronic hepatitis B (CHB) shown in FIG. 11 and hepatitis type C virus carriers with persistently normal ALT (CNALT) shown in FIG. 12 are all 1.000 in AUC, thereby confirming that they can be identified at an extremely high accuracy according to the method of the present invention.

### 5. Evaluation of γ-Glu-X Peptides in Other Diseases

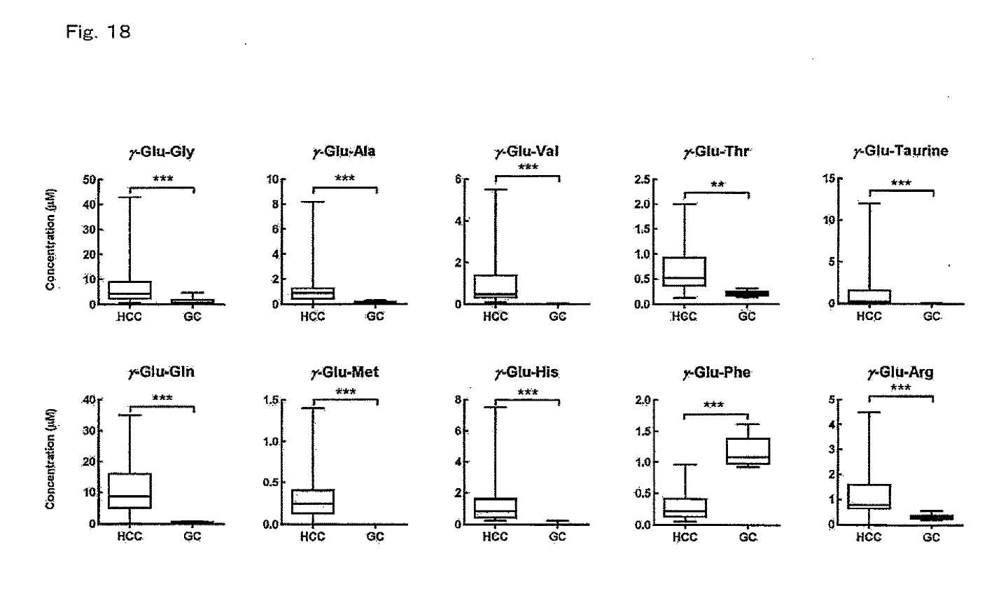

We determined whether γ-Glu-X peptides were increased in other diseases. FIG. 18 shows the concentrations of γ-Glu-X peptides in the sera from a hepatocellular carcinoma patients (HCC) and a gastric cancer patients (GC). In the gastric cancer patients (GC), the concentrations of γ-Glu-X peptides were equivalent to those of normal persons (C) and there was no increase in the γ-Glu-X peptides which was seen in the hepatocellular carcinoma patients (HCC). (Note: it is reported that infection with Helicobacter pylori causes gastric cancer and suppresses oxidative stress (reference 10). It is suspected that the concentrations of γ-Glu-X peptides are low since gastric cancer is not exposed to oxidative stress).

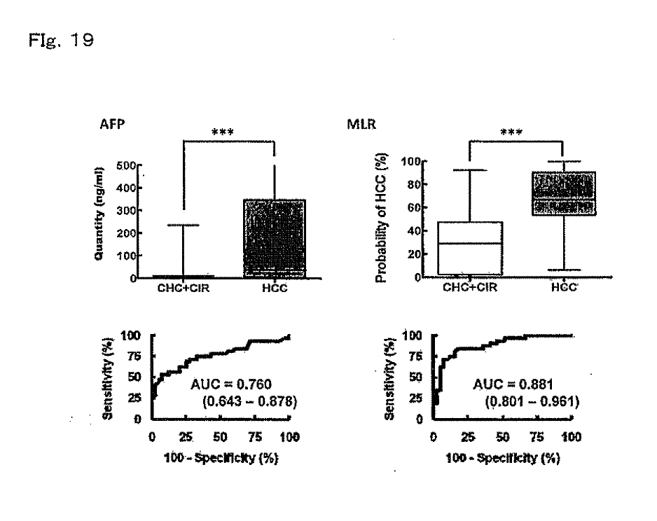

FIG. 19 shows boxplots of α-fetoprotein (AFP) and MLR as well as receiver operating curves (ROC) for distinguishing patients with hepatocellular carcinoma (HCC) (the number of patients: 32) from patients with chronic hepatitis C (CHC) (the number of patients: 35) and patients with cirrhosis type C (CIR) (the number of patients: 18). In the MLR model, there used are γ-Glu-Ala, γ-Glu-Citrulline, γ-Glu-Thr and γ-Glu-Phe. And, p-values of Mann-Whitney tests were less than 0.0001 both in AFP and MLR. In the boxplot of AFP, 6 plots in a group of patients with HCC were outside of the plot (>500 ng/mL). A value in the ROC indicates an area equal to or lower than the ROC and 95% confidence interval thereof.

Further, diabetics are also low in blood concentrations of γ-Glu-X peptides, and γ-Glu-X peptides in the blood were increased in a liver disease-specific manner.

### 6. Elucidation of Biosynthesis Mechanism of γ-Glu-X Peptides

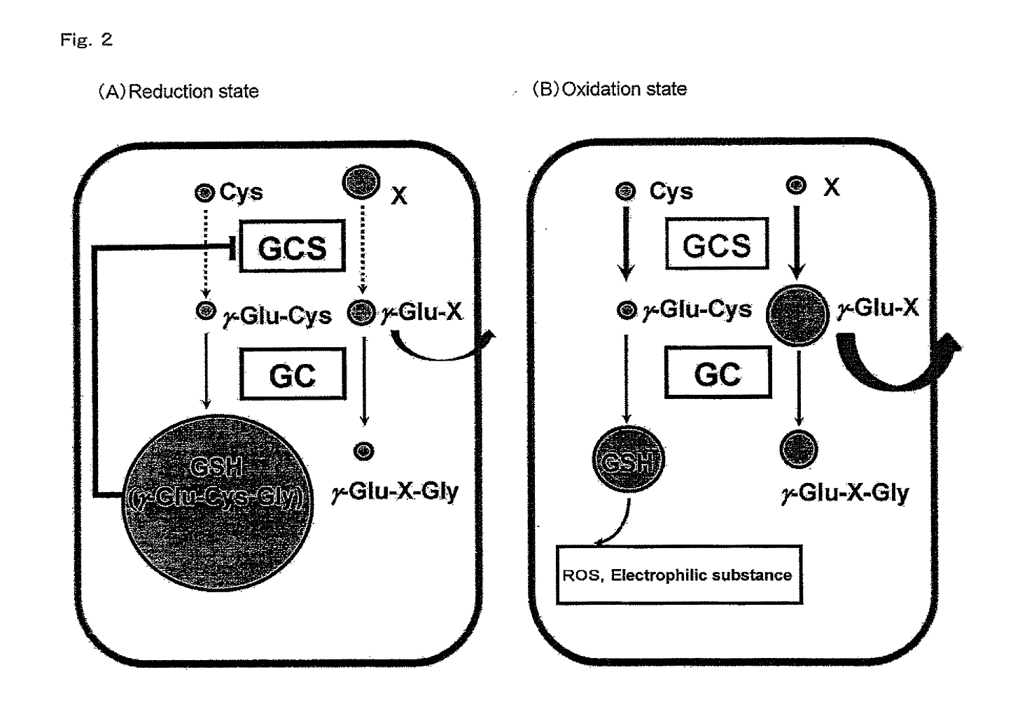

The biosynthesis mechanism of γ-Glu-x peptides was elucidated using a mouse. As shown in FIG. 2(B), the following biosynthesis mechanism of γ-Glu-X peptides was found. That is, the oxidative stress condition caused by active oxygen and electrophiles causes depletion of glutathione, which is used for removing these substances, and this depletion leads to activation of γ-glutamyl cysteine synthase (GCS). Then, γ-Glu-X dipeptides and γ-Glu-X-Gly tripeptides are biosynthesized by using various amino acids as substrates (starting materials). The experimental procedures are described below.

a. Administration of GCS Inhibitor, BSO, and GCS Activator, DEM, to Mice

Male mice were fasted overnight and anesthetized by an intraperitoneal injection of pentobarbital sodium (60 mg/Kg of body weight). Then, the mice were intraperitoneally injected with BSO, which is aγ-glutamyl cysteine synthase (GCS) inhibitor, and diethylmaleate (DEM), which is an electrophile (GCS activator), both at a dose of 4 mmol/kg of body weight (888 mg of BSO, 688 mg of DEM). The mouse as a healthy subject was intraperitoneally injected with physiological saline. Liver specimens (about 300 mg) were excised from the mice 1 hour after administration (5 times each).

b. Extraction of Metabolites from the Livers

The liver specimens (about 300 mg) excised from the mice were immediately placed in 1 ml of methanol containing an internal standard substance and were homogenized to inactivate an enzyme, thereby stopping enhancement of metabolism. After 500 μl of pure water was added to the mixture, 300 μl of solution was taken out. To this solution, 200 μl of chloroform was added. After thorough stirring, the solution was further centrifuged at 15000 rpm for 15 minutes at 4° C. After allowing to stand, 300 μl of separated water-methanol phase was passed through a 5 kDa molecular weight cutoff filter for centrifugal ultrafiltration to deproteinize it. The filtrate was lyophilized and 50 μl of Milli-Q water was added thereto. The mixture was subjected to a CE-TOFMS measurement.

c. Identification Results of γ-Glu-X Peptides and γ-Glu-X-Gly Peptides as Biomarkers that are Indicators of Oxidative Stress

FIG. 11 shows a part of the measurement results of amino acids, γ-Glu-X peptides, and γ-Glu-X-Gly peptides in the livers and the sera from the mice after administration of physiological saline (a healthy subject), BSO, or DEM. The left part of FIG. 11 shows the quantification results of amino acids (X), γ-Glu-X peptides, and γ-Glu-X-Gly peptides detected in the livers from the mice that received each of the reagents. The right part of FIG. 11 shows the quantification results of amino acids (X), γ-Glu-X peptides, and γ-Glu-X-Gly peptides in the sera from the mice.

For example, the graphs on the top show the results of Cys, γ-Glu-Cys, and γ-Glu-Cys-Gly (glutathione). The amount of glutathione in the livers was decreased rapidly in the BSO-administered mouse and the DEM-administered mouse compared to the healthy mouse (In the BSO-administered mouse, glutathione is decreased because of inhibition of γ-glutamyl cysteine synthase. In the electrophilic DEM-administered mouse, glutathione is decreased since it is consumed for detoxication). No glutathione related substances were detected in the sera.

The method described below was used to confirm that the detected γ-Glu-X peptides and γ-Glu-X-Gly peptides were synthesized in a glutathione-biosynthesis pathway. As shown in FIG. 2, if these peptides were synthesized in the glutathione-biosynthesis pathway, the γ-Glu-X peptides and γ-Glu-X-Gly peptides in the liver should be decreased by administration of BSO (since γ-glutamyl cysteine synthase is inhibited) and be increased by administration of electrophilic DEM (since γ-glutamyl cysteine synthase is activated), compared to the normal person.

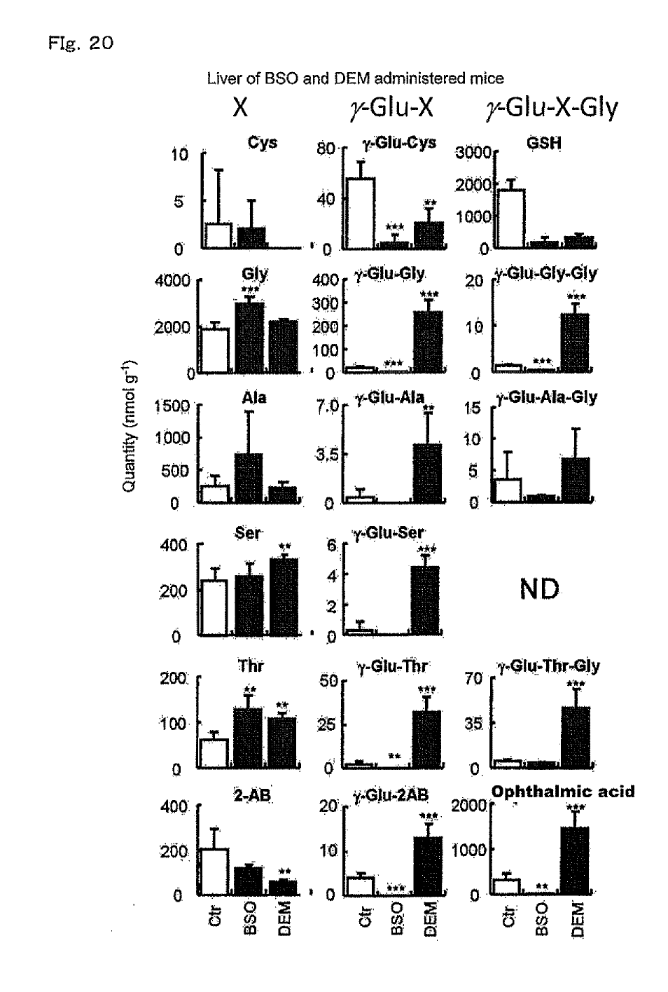

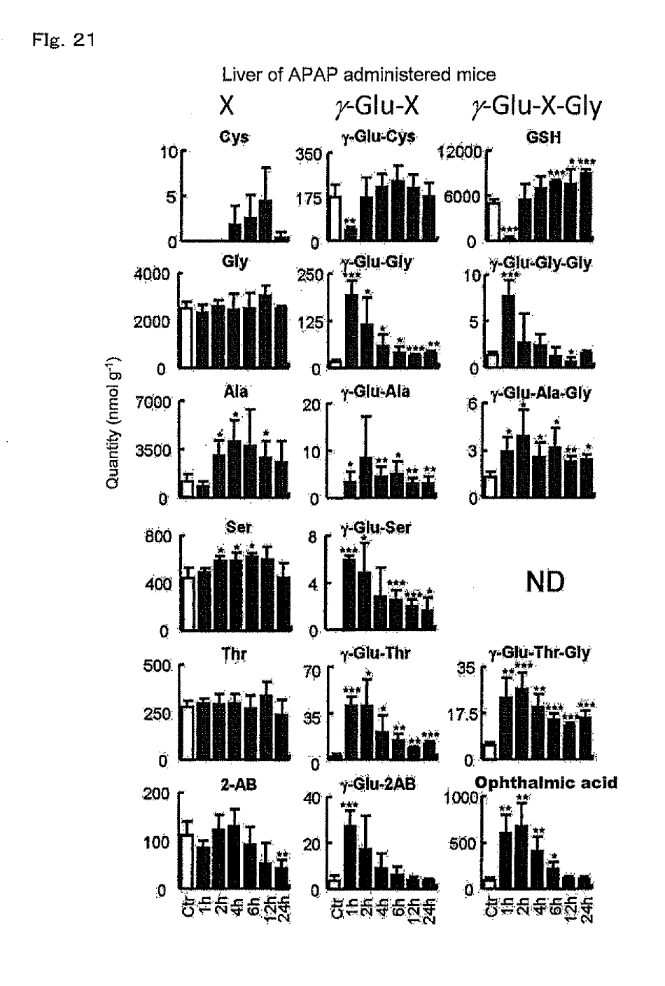

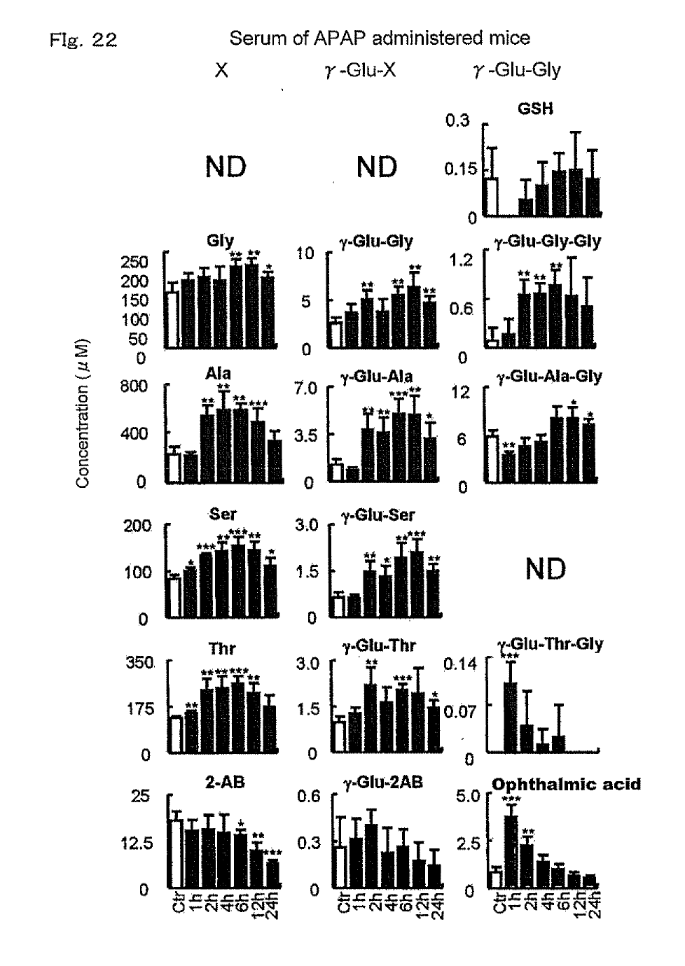

FIG. 20, FIG. 21 and FIG. 22 show respectively measurement results of the livers obtained from the mice to which BSO and DEM are administered, measurement results of the livers obtained from the mice to which acetaminophen (APAP) is administered and measurement results of the sera obtained from the mice to which acetaminophen (APAP) is administered. As shown in FIGS. 20-22, γ-Glu-X peptidic substances and γ-Glu-X-Gly peptidic substances in the liver other than glutathione-related γ-Glu-Cys, GSH, and γ-Glu-Ser-Gly were decreased by administration of BSO and were increased by administration of DEM compared to the normal person. Therefore, it was confirmed that the γ-Glu-X peptidic substances and γ-Glu-X-Gly peptidic substances were certainly generated in the glutathione-biosynthesis pathway.

In short, it was found that these γ-Glu-X peptides and γ-Glu-X-Gly peptides were biosynthesized in the liver when glutathione was decreased because of oxidative stress caused by active oxygen, electrophiles, and the like.

d. Tracing of the Biosynthesis Pathway Using Threonine Isotope

Furthermore, 13C, 15N isotope-labeled threonine (Thr) was administered to a mouse intraperitoneally, and APAP, which produces an electrophile and thereby gives oxidative stress, was given to the mouse. γ-Glu-Thr and γ-Glu-Thr-Gly containing 13C, 15N-labeled Thr were detected in the murine liver and it was confirmed that γ-Glu-Thr and γ-Glu-Thr-Gly were certainly biosynthesized from Thr under the oxidative stress condition.

On the other hand, regarding the substances in the murine serum, γ-Glu-X peptides and γ-Glu-X-Gly peptides that were decreased by administration of BSO and were increased by administration of an electronic substance, DEM, compared to the normal state were only γ-Glu-2AB and ophthalmic acid (γ-Glu-2AB-Gly) (FIG. 22). Therefore, it is expected that, in the case of a mouse, only γ-Glu-2AB and ophthalmic acid are increased in the blood when glutathione is decreased because of the oxidative stress caused by electrophiles or the like.

However, when the sera from patients with various types of hepatitis were measured, the concentrations of the other γ-Glu-X peptides were higher than those of γ-Glu-2AB and ophthalmic acid. This difference occurs presumably since the substrate concentration, the substrate specificity and activity of a metabolic enzyme, the type, function, and level of expression of a transporter, and the like are different between organism species.

The diagnosis method according to the present invention based on the measurement of γ-Glu-X peptides in serum can also apply to diagnosis of various liver injuries such as hepatic cirrhosis, a nonalcoholic fatty liver disease (NAFLD), nonalcoholic hepatitis (NASH), and simple steatosis (SS).

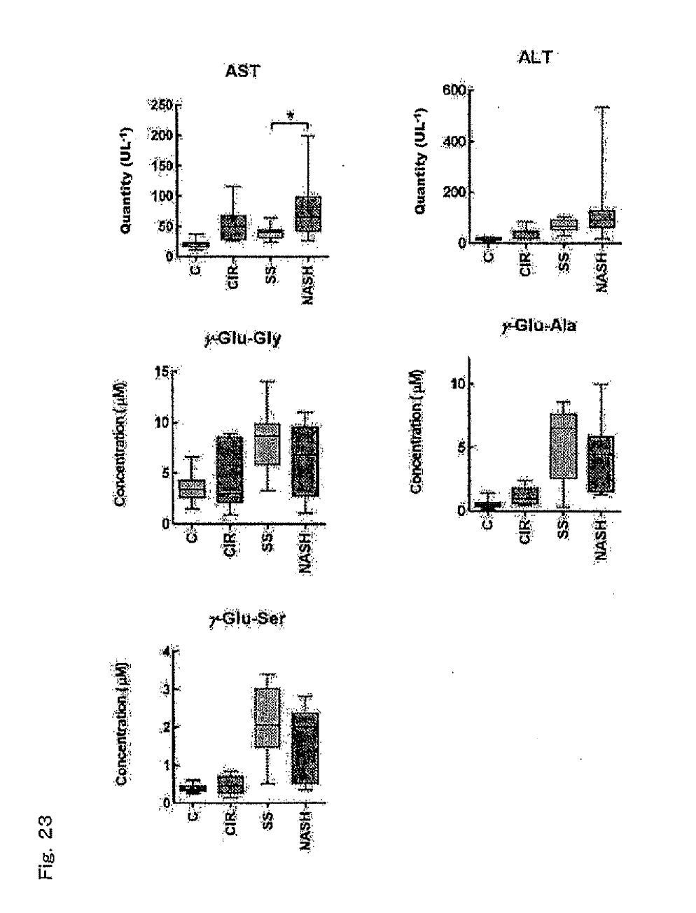

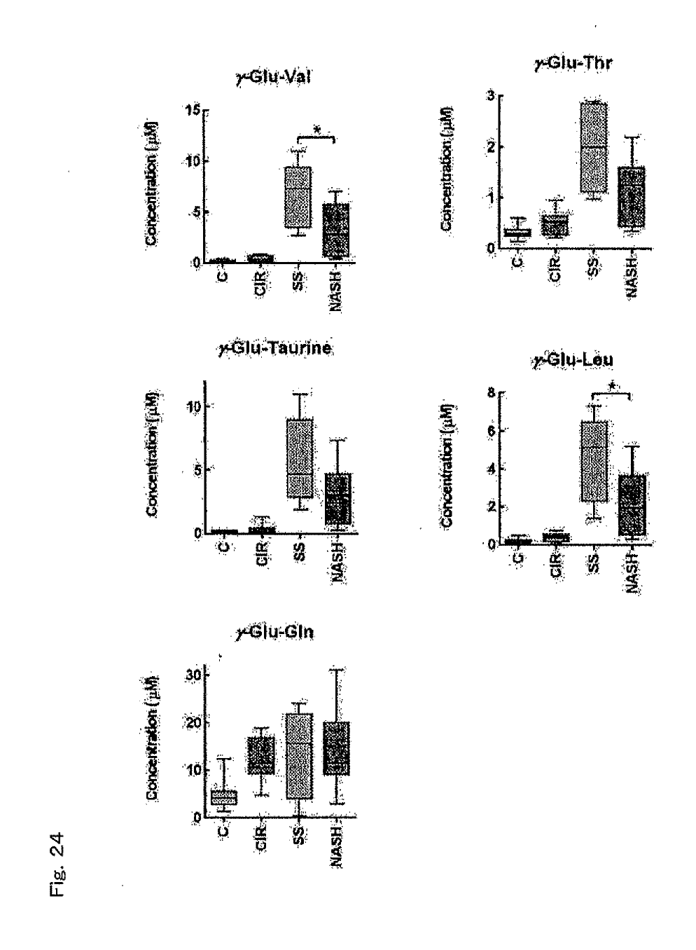

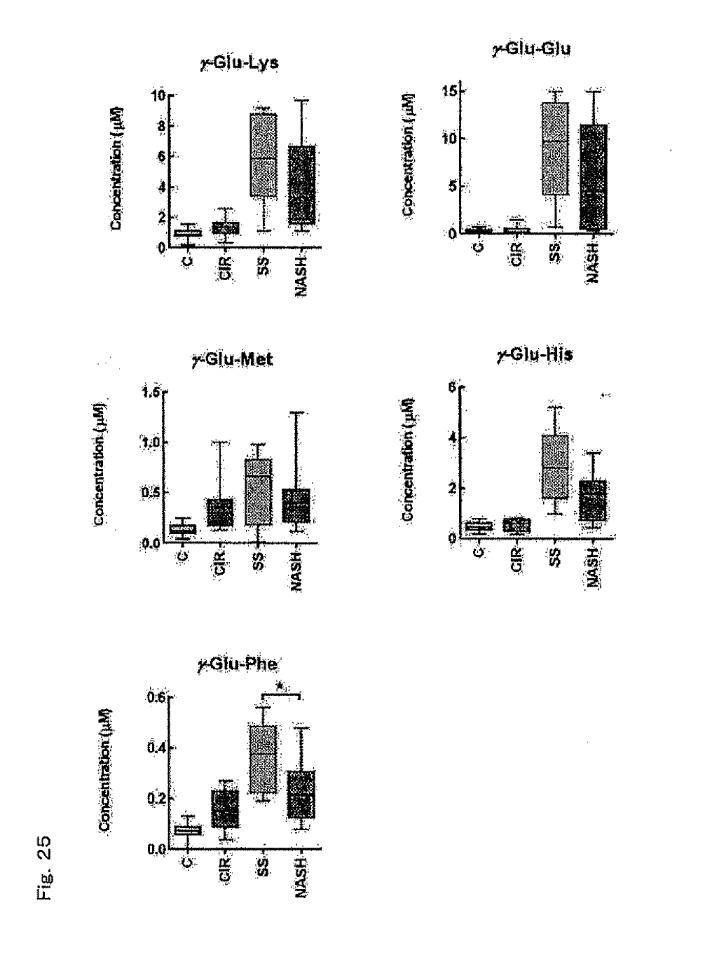

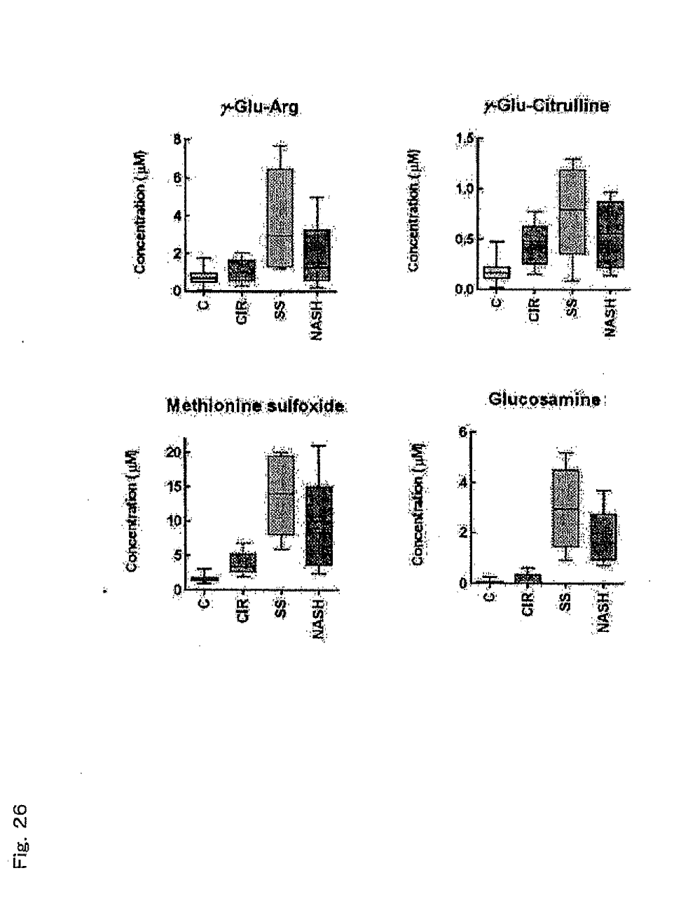

FIG. 23 to FIG. 26 show data on normal persons (C)/patients with cirrhosis type C (CIR), patients with simple steatosis (SS) and patients with non-alcoholic steatohepatitis (NASH). It is recognized that simple steatosis (SS) can be distinguished from non-alcoholic steatohepatitis (NASH) with reference to AST, γ-Glu-Val, γ-Glu-Leu and γ-Glu-Phe, each p-value of which is lower than 0.05, as indicated with an asterisk.

Here, an example is given of combination of AST, ALT, and γ-Glu-X peptides which are marker candidates of various types of liver disorders, to which the present invention shall not be restricted. In the future, more samples will be studied in detail, by which a combination of types of biomarker candidates may be changed.

Although a LC-MS method was used for measuring γ-Glu-X peptides in serum in this study, measuring methods are not limited and any analysis method can be used for the measurement. Exemplary measuring methods include gas chromatography (GC), liquid chromatography (LC), capillary electrophoresis (CE), chip LC, chip CE, and GC-MS, LC-MS, and CE-MS methods that combine them with a mass spectrometer (MS), various measuring methods using MS alone, an NMR method, a method of measuring γ-Glu-X peptides after their derivatization to fluorescent substances or UV absorbing substances, a measuring method involving producing an antibody and performing an ELISA method using the antibody, and the like.

The method for assaying a pharmaceutical preparation according to the present invention is a method for measuring the concentration of the marker of the present invention in a mammal, by using the blood collected from the mammal that received the pharmaceutical preparation and the blood collected from the mammal that did not receive the pharmaceutical preparation. Although the mammal from which blood used for this assay method is collected is not particularly limited, a mammal whose blood contains at least one of the above-mentioned markers is preferred. Rodents such as a mouse and a rat, a human, a monkey, and a dog are more preferred.

In the above-mentioned method, when the efficacy of a pharmaceutical preparation as a therapeutic agent for a toxic electrophile and active oxygen (oxidative stress) is examined, the targeted disease to which the pharmaceutical preparation is applied is not limited as long as it is a disease caused by oxidative stress. The targeted disease is similar to the diseases as described above for which a marker is used. Furthermore, when the strength of the oxidative stress generated by administration of a pharmaceutical preparation is examined, the type of the pharmaceutical preparation is not limited in any way. For example, pharmaceutical preparations may include a harmful drug.

The purposes of the assay method according to the present invention are to examine the efficacy of a pharmaceutical preparation as a therapeutic agent for a disease caused by oxidative stress and to examine the strength of the oxidative stress generated by administration of the pharmaceutical preparation; however, in practice, the method may be used in various situations. Although representative examples of use of the assay method according to the present invention are described below, the inventive assay method is not limited thereto.

### (1) Assay of Efficacy as Therapeutic Agent

Hereinbelow, exemplary uses in hepatitis of the assay method according to the present invention are described; however, the assay method is not limited to these examples.

### (1-1) Medicinal Effect Assay in Specific Individual

For example, the assay method according to the present invention can be used to determine if a therapeutic agent for hepatitis is effective in treating the hepatitis of a specific patient. First, blood is collected from a patient suffering from hepatitis before and after administration of the therapeutic agent for hepatitis to the patient. Then, the concentration of a hepatitis diagnostic marker in the blood is measured. The thus obtained marker concentrations in the blood before and after administration of the therapeutic agent for hepatitis are compared. When the marker concentration in the blood after administration of the therapeutic agent for hepatitis is significantly lower compared to that before administration, one can determine that the therapeutic agent for hepatitis is effective in treating the hepatitis of the patient.

### (1-2) General Medicinal Effect Assay

Furthermore, it is also possible to assay general efficacy of the pharmaceutical preparation as a therapeutic agent for hepatitis by applying the assay method according to the present invention to a plurality of human individuals.

For example, by comparing the concentrations of a hepatitis diagnostic marker before and after administration of a therapeutic agent for hepatitis in a plurality of humans suffering from hepatitis, one can examine a universal effect of the substance as a therapeutic agent.

Alternatively, in another embodiment, the effect as a pharmaceutical preparation may be compared between two groups. First, patients suffering from hepatitis are divided into two groups. The patients of one group receive a therapeutic agent for hepatitis, while the patients of the other group do not receive the therapeutic agent or receive a placebo. Blood is collected from the patients of these two groups. Then, the concentration of a hepatitis diagnostic marker in the blood is measured. Furthermore, the concentrations of the marker in the blood obtained by this measurement are compared between the two groups.

Herein, a “group” may include only a single individual or a plurality of individuals, and the number of the individuals of the two groups may be the same or different. The blood collected from the individuals in the same group may be pooled and the marker concentration in the pooled blood may be measured. However, it is preferable to measure the marker concentration in each individual's blood separately.

The comparison of the marker concentrations among groups including a plurality of blood samples may be performed by comparing pairs of blood samples or by comparing among the groups a cumulative total or a mean value of the marker concentrations obtained from the plurality of blood samples belonging to the same group. The plurality of blood samples are for example, blood samples before and after or in the presence or absence of administration of a pharmaceutical preparation. This comparison may be performed using any statistical method well known to those of ordinary skill in the art. As a result of such comparisons, when the marker concentration in the blood after administration of a therapeutic agent is significantly lower compared to that before administration or when the marker concentration in the blood from the group receiving the therapeutic agent is significantly lower compared to that of the group receiving no therapeutic agent, one can determine that the therapeutic agent is effective for the treatment of hepatitis. Moreover, one can determine the level of efficacy by the degree of decrease.

In this way, screening for a therapeutic agent for hepatitis can be achieved by assaying its general efficacy as a therapeutic agent for hepatitis. Furthermore, the strength of medicinal effect of each therapeutic agent for hepatitis can be examined by using a plurality of therapeutic agents for hepatitis and examining therapeutic effects of different concentrations of each of the therapeutic agents for hepatitis, and comparing their different medicinal effects dependent on their concentrations.

### (2) Assay of the Strength of Oxidative Stress

Strong oxidative stress leads to a strong expression of a side effect. Therefore, exemplary uses of the assay method according to the present invention for the side effect of a pharmaceutical preparation is described below by way of example; however, the inventive assay method is not limited to these examples.

### (2-1) Assay of the Strength of Side Effect in Specific Individual

The assay method according to the present invention can be used to determine if a certain pharmaceutical preparation brings a side effect to a specific mammalian individual. First, blood is collected from the individual before and after administration of the therapeutic pharmaceutical preparation to the individual. Then, the concentration of an oxidative stress assay marker in the blood is measured. The thus obtained marker concentrations in the blood before and after administration of the pharmaceutical preparation are compared. When the marker concentration in the blood after administration of the pharmaceutical preparation is significantly higher compared to that before administration, one can determine that the administered pharmaceutical preparation caused oxidative stress in the individual, causing a side effect in the individual.

### (2-2) Assay of General Strength of a Side Effect

Furthermore, it is also possible to assay a general strength of a side of feet of a certain pharmaceutical preparation by applying the assay method according to the present invention to a plurality of mammalian individuals.

For example, it is possible to examine a general strength of a side effect of the pharmaceutical preparation by comparing the concentrations of an oxidative stress assay marker before and after administration of the pharmaceutical preparation for treating a disease in a plurality of individuals suffering from the disease.

Alternatively, in another embodiment, the strength of a side effect can be compared between two groups. First, mammals suffering from a disease are divided into two groups. The individuals of one group receive the pharmaceutical preparation for treating the disease, while the individuals of the other group do not receive the pharmaceutical preparation or receive a placebo. Blood is collected from the individuals of these two groups. Then, the concentration of an oxidative stress assay marker in the blood is measured. Furthermore, the concentrations of the markers in the blood obtained by this measurement are compared between the two groups. Herein, a “group” may include only a single individual or a plurality of individuals, and the number of the individuals of the two groups may be the same or different. The blood collected from the individuals in the same group may be pooled and the marker concentration in the pooled blood may be measured. However, it is preferable to measure the marker concentration in each individual's blood separately.

The comparison of the marker concentrations among groups may be performed by comparing pairs of blood samples or by comparing among the groups a cumulative total or a mean value of the marker concentrations obtained from the plurality of blood samples belonging to the same group. This comparison may be performed using any statistical method well known to those of ordinary skill in the art. As a result of such comparisons, when the marker concentration in the blood after administration of the pharmaceutical preparation is significantly higher compared to that before administration or when the marker concentration in the blood from the group receiving the pharmaceutical preparation is significantly higher compared to that of the group receiving no pharmaceutical preparation, one can determine that the pharmaceutical preparation has a side effect.

In this way, screening for a pharmaceutical preparation with a weak side effect can be achieved by assaying the strength of the side effect of the pharmaceutical preparation. Furthermore, the suitability of each pharmaceutical preparation as a therapeutic agent can be compared by using a plurality of pharmaceutical preparations and examining the pharmaceutical effects of different concentrations of each of the pharmaceutical preparations, and comparing their different side effects dependent on their concentrations.

As described above, the oxidative stress assay marker of the present invention can be used for the assay of a pharmaceutical preparation for treating a liver disease or the assay of the strength of a side effect of a pharmaceutical preparation, diagnosis of a disease, and the like. On that occasion, the assay accuracy and diagnosis accuracy can be increased by using a plurality of markers. Assay methods and diagnosis methods other than the marker of the present invention may also be combined.

### INDUSTRIAL APPLICABILITY

The present invention discovered a biomarker, a γ-Glu-X peptide, which indicates the depletion of glutathione caused by oxidative stress that was generated in a living organism. The γ-Glu-X peptide is not only useful as a rapid screening method for patients with various liver injuries, but also can serve as a marker for detecting oxidative stress in a living organism in a wide range of life science fields.

1. A liver disease marker for detecting an oxidative stress in a mammalian tissues and wherein
the marker is a combination of a plurality of γ-Glu-X (X represents an amino acid or an amine) peptides and at least one of glucosamine, methionine sulfoxide, AST and ALT.

2. The liver disease marker according to claim 1, and wherein
the marker is a liver disease marker for identifying a normal person which is a combination including at least glucosamine, γ-Glu-Ala, methionine sulfoxide, γ-Glu-Leu, γ-Glu-Val, AST, ALT, γ-Glu-Phe, γ-Glu-Met and γ-Glu-Gln.

3. A liver disease marker for identifying drug-induced liver injury which is a combination including at least γ-Glu-Taurine, γ-Glu-Leu, γ-Glu-Glu, γ-Glu-Gly, γ-Glu-Arg, γ-Glu-Ser, γ-Glu-Phe, γ-Glu-Met and γ-Glu-Citrulline.

4. The liver disease marker according to claim 1, and wherein
the marker is a liver disease marker for identifying an asymptomatic hepatitis B carrier which is a combination including at least γ-Glu-Taurine, γ-Glu-Ala, γ-Glu-Leu, γ-Glu-Val, AST, γ-Glu-Lys, γ-Glu-Arg, γ-Glu-Met and γ-Glu-Gln.

5. The liver disease marker according to claim 1, and wherein
the marker is a liver disease maker for identifying chronic hepatitis B which is a combination including at least γ-Glu-Ala, methionine sulfoxide, γ-Glu-Leu, γ-Glu-Glu, AST, ALT, γ-Glu-Arg, γ-Glu-Ser, γ-Glu-His, γ-Glu-Phe, γ-Glu-Met and γ-Glu-Citrulline.

6. The liver disease marker according to claim 1, and wherein
the marker is a liver disease marker for identifying a hepatitis C virus carrier with persistently normal ALT which is a combination including at least glucosamine, γ-Glu-Leu, γ-Glu-Val, AST, γ-Glu-Gly, γ-Glu-Gln and γ-Glu-Citrulline.

7. The liver disease marker according to claim 1, and wherein
the marker is a liver disease marker for identifying chronic hepatitis C which is a combination including at least glucosamine, γ-Glu-Lys and γ-Glu-His.

8. The liver disease marker according to claim 1, and wherein
the marker is a liver disease marker for identifying cirrhosis type C which is a combination including at least glucosamine, methionine sulfoxide, γ-Glu-Leu, γ-Glu-Val, γ-Glu-Glu, γ-Glu-Gly, γ-Glu-Met, γ-Glu-Gln and γ-Glu-Citrulline.

9. A liver disease marker for identifying hepatocellular carcinoma which is a combination including at least γ-Glu-Taurine, γ-Glu-Glu, γ-Glu-Gly, γ-Glu-Ser and γ-Glu-Citrulline.

10. The liver disease marker according to claim 1, and wherein
the marker is a liver disease marker for identifying simple steatosis which is a combination including at least γ-Glu-Taurine, γ-Glu-Ala, γ-Glu-Leu, γ-Glu-Val, γ-Glu-Glu, AST, ALT, γ-Glu-Thr and γ-Glu-Gln.

11. The liver disease marker according to claim 1, and wherein
the marker is a liver disease marker for identifying non-alcoholic steatohepatitis which is a combination including at least glucosamine, γ-Glu-Ala, γ-Glu-Val, γ-Glu-Gly, γ-Glu-Gln and γ-Glu-Citrulline.

12. A method for measuring a liver disease marker in which a plurality of γ-Glu-X (X represents an amino acid or an amine) peptides and at least one of lucosamine, methionine sulfoxide AST and ALT in a sample are measured as the liver disease marker.

13. An apparatus for measuring a liver disease marker comprising:
means for preparing a test sample suitable for analysis from a sample; and
analysis means for measuring a plurality of γ-Glu-X (X represents an amino acid or an amine) peptides and at least one of glucosamine, methionine sulfoxide, AST and ALT in the test sample as the liver disease marker.

14. A method for assaying a pharmaceutical preparation comprising the steps of:
measuring a concentration of the liver disease marker according to claim 1 in blood samples collected before and after administration of a drug; and
comparing the measurement results of the blood samples collected before administration of the drug with the measurement results of the blood samples collected after administration of the drug.

15. A method for assaying a pharmaceutical preparation comprising the steps of:
measuring a concentration of the liver disease marker according to claim 1 in blood samples collected from a first group composed of one or more individuals to which a drug is administered and in blood samples collected from a second group composed of one or more individuals to which the drug is not administered; and
comparing the concentration of the liver disease marker measured in the first group with the concentration thereof measured in the second group.
